(19) United States
(12) Patent Application Publication
(10) Pub. No.: US 2020/0345686 A1
(43) Pub. Date: Nov. 5, 2020

(54) COMPOSITIONS AND METHODS FOR TREATING SKIN AND NEUROPATHIC CONDITIONS AND DISORDERS

(71) Applicant: VCP IP HOLDINGS, LLC, LIMITED LIABILITY COMPANY DELAWARE, CHICAGO, IL (US)

(72) Inventors: ANDREA BAILLO, CHICAGO, IL (US); ALEXANDRA DePALMA, CHICAGO, IL (US)

(73) Assignee: VCP IP HOLDINGS, LLC, LIMITED LIABILITY COMPANY DELAWARE, CHICAGO, IL (US)

(21) Appl. No.: 16/860,270

(22) Filed: Apr. 28, 2020

Related U.S. Application Data
(60) Provisional application No. 62/841,198, filed on Apr. 30, 2019.
(60) Provisional application No. 62/947,803, filed on Dec. 13, 2019.

(51) Int. Cl. A61K 31/352; A61K 31/05; A61K 31/165; A61K 31/365

(52) U.S. Cl. CPC A61K 31/352; A61K 31/05; A61K 9/0014; A61K 31/365; A61K 31/165

(57) ABSTRACT

Described herein are compositions for treating dermatological conditions, such as skin conditions, as well as neuropathological conditions and disorders, particularly those requiring relief from pain and physical discomfort. The compositions as described may be topically or transdermally administered. In some aspects, the compositions and products comprise a pharmaceutically acceptable amount of at least one analgesic component and a cannabis component and are useful to relieve acute or chronic pain, inflammation, and the like, following application.

### CROSS REFERENCE TO RELATED APPLICATIONS

This application claims priority to and benefit of U.S. Provisional Application No. 62/947,803, filed on Dec. 13, 2019 and U.S. Provisional Application No. 62/841,198, filed on Apr. 30, 2019, the contents of each of which are incorporated by reference herein in their entireties.

### BACKGROUND

Because of its exposure and surface area, the skin is routinely subjected to a variety of external and internal insults that detrimentally affect its condition and appearance. Moreover, pathological conditions, disorders and diseases that affect the skin can cause major discomfort and pain, both in general and, in particular, as individuals age. During the aging process and as a result of certain conditions and disorders, such as neurological conditions, disorders, or symptoms, the appearance, texture and overall condition of the skin, as well as its function, can deteriorate both acutely and chronically, for example, from exposure to various environmental conditions, such as ultraviolet radiation, and from disease, such as inflammatory diseases.

Compositions and methods of providing directed treatments and therapies for skin conditions, aging skin, damaged skin, neurological conditions and disorders, and the pain associated with such conditions and disorders are needed to effectively treat, alleviate and provide relief to afflicted individuals. The compositions and methods described herein address such needs.

### SUMMARY OF THE EMBODIMENTS

In an aspect as described and featured herein, a composition for treating or healing a skin or neurological condition or disorder is provided, in which the composition comprises a pharmaceutically effective amount of at least one analgesic component and at least one cannabis component, and a pharmaceutically acceptable carrier, excipient, or diluent. In an embodiment, the at least one analgesic component comprises one or more of capsaicin, sesquiterpene lactones and eucalyptol. In another embodiment, the at least one cannabis component is a cannabinoid, a cannabinol, or a cannabidiol. In another embodiment, the cannabinoid is (−)-trans-cannabidiol (CBD) or tetrahydrocannabinol (THC). In an embodiment, the ratio of CBD:THC in the composition is 5:1 or 1:3. In an embodiment, the composition further comprises Vitamin A, Vitamin B1, Vitamin B2, Vitamin B6, Vitamin C, Vitamin E, terpinen-4-ol and 1,8 cineole, carotenoids, triterpenic alcohols, or a combination thereof. In an embodiment, the composition is in the form of a salve. In an embodiment, the composition further comprises one or more of isopropyl palmitate, soy lecithin, oleic acid, shea nut butter, beeswax, sunflower oil, lanolin, olive oil, antimicrobials, or antivirals. In a particular embodiment, the composition comprises seabuckthorn oil, capsicum oil, arnica, tea tree, eucalyptus, calendula, vitapherol T-50, and Optiphen ND.

In an aspect, a method of treating a suffering from, or at risk of, a skin condition, acute injury, inflammation, pain, skin allergy, muscle spasms, or skin aging is provided in which the method comprises administering to the subject an effective amount of the composition of the above-delineated aspect and embodiments. In an embodiment of the method, the subject suffers from shingles, arthritis, fibromyalgia, migraine, neuropathy, back pain, sprains, muscle pain, cartilage pain, acne, insect bite, skin rash, skin wound, or a combination thereof.

In another aspect, a composition for treating or healing a skin or neurological condition or disorder is provided, in which the composition comprises a pharmaceutically effective amount of at least one antioxidant component, an avenanthramide component and at least one cannabis component, and a pharmaceutically acceptable carrier, excipient, or diluent. In an embodiment, the at least one cannabis component is a cannabinoid, a cannabinol, or a cannabidiol. In an embodiment, the cannabinoid is (−)-trans-cannabidiol (CBD) or tetrahydrocannabinol (THC). In an embodiment, the ratio of CBD:THC in the composition is 1:1. In an embodiment, the composition further comprises sesame oil, hydrolyzed oats, D-orientine S, collagen, aloe vera, polyphenols, Vitamin E, Vitamin B3, terpenes, flavonoids, antimicrobials, or a combination thereof. In an embodiment, the composition is in the form of a liniment. In a particular embodiment, the composition further comprises one or more of isopropyl palmitate, soy lecithin, oleic acid, shea nut butter, beeswax, jojoba oil, ceteryl alcohol, glyceryl stearate, and Optiphen ND.

In an aspect, a method of treating a subject suffering from, or at risk of, a skin condition, damaged skin, inflammation, aging skin, wrinkling skin, sunburn, pain, or a combination thereof is provided, in which the method comprises administering to the subject an effective amount of the composition of the above-delineated aspect and embodiments. In an embodiment, the subject is suffering from acne, acne vulgaris, psoriasis, eczema, hyperpigmentation, or a combination thereof.

In an aspect of any of the above-delineated methods, the composition is administered by topical application. In an aspect of any of the above-delineated methods, the composition is administered transdermally. In an aspect of any of the above-delineated methods. the composition is administered prophylactically or therapeutically.

In another aspect, a composition for topical or transdermal application to the skin or body is provided, the composition comprising an effective amount of a cannabinoid selected from THC, THCa, THCV, CBN, CBC, CBD, CBG, or a combination thereof, and a pharmaceutically acceptable carrier, excipient, or diluent. In an embodiment of the composition, the cannabinoids comprise THC, THCa, THCV, CBN, CBC and CBG. In an embodiment, the cannabinoid is present in an amount of about 0.2 mg to about 49 mg. In an embodiment, the composition is a moisturizer or lubricant. In another embodiment of the composition, the cannabinoids comprise THC, CBD and CBG. In an embodiment, the cannabinoid is present in an amount of about 1 mg to about 18 mg (w/w). In an embodiment, the composition is a salve. In yet another embodiment of the composition, the cannabinoids comprise THC and CBG. In an embodiment, the cannabinoid is present in an amount of about 3 mg to about 117 mg. In an embodiment, the composition is a lotion. In some cases, the composition, especially a salve composition as delineated above, further comprises a terpene or cannabis derived terpene. In an embodiment, the terpene or cannabis derived terpene is selected from α-pinene, ß-myrcene, D-limonene, terpinolene, or a combination thereof. In an embodiment, the salve composition further comprises shea nut butter, beeswax, sunflower oil, lanolin, olive oil, seabuckthorn oil, capsicum oil, oleic acid, arnica, tea tree, eucalyptus, isopropyl palmitate, soy lecithin, optiphen ND, calendula, medical cannabis, vitapherol T-50, or a combination thereof. In an embodiment, the lotion composition further comprises Shea butter, Sesame oil, Jojoba oil, Hydrolyzed oats, Ceteryl alcohol, D'Orientine S, Syn Coll, Oleic acid, Olivem, Aloe vera liquid, Optiphen ND, Antioxidant complex T5, Glyceryl stearate, Vitapherole T-50, Niacinamide, Isopropyl palmitate, Soy lecithin, Medical cannabis, Palmarosa, Grapefruit, or a combination thereof. In an embodiment, the moisturizer or lubricant composition further comprises medium-chain triglyceride (MCT) oil, aloe vera and medical cannabis.

In another aspect, a method of treating a subject suffering from or at risk of from a skin condition, skin injury, damaged skin, pain, inflammation, or a combination thereof, is provided, in which the method comprises administering to the subject an effective amount of the composition as delineated above. In an embodiment of the method, the composition is in the form of a lotion, lubricant, or salve. In an embodiment of the method, the composition is administered by topical application. In an embodiment of the method, the composition is administered transdermally. In an embodiment or the method, the composition is administered prophylactically or therapeutically.

In an aspect of any of the above-delineated methods in which the subject is suffering from or at risk of pain, the pain is associated with a disease or disorder selected from arthritis, Rheumatoid arthritis, diabetic neuropathy, sciatica, Ankylosing Spondylitis, or Neuromyelitis Optica Spectrum Disorder.

Compositions, products, or articles described herein were isolated or otherwise manufactured in connection with the examples provided below. Other features and advantages of the embodiments will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

An “active ingredient,” “active component,” or “active” is broadly construed to include any ingredient considered to have an effect, such as a therapeutic effect, when delivered or administered to a subject in need thereof. In an embodiment, an active ingredient may be regulated by a drug agency or authority, e.g., CDER, FDA, EMEA, TAG, and the like. In an embodiment, an active ingredient may be one or more cannabinoids, such as THC or CBD, or others as described herein. Active ingredients may act systemically, e.g., following certain types of administration (e.g., intravenous, subcutaneous, topical, transdermal, and the like), or upon swallowing, or they may act locally (e.g., at an administration site), or when present in the mouth or under the tongue.

By “agent” is meant any small molecule chemical compound, ingredient, component, antibody, nucleic acid molecule, or polypeptide, or fragments thereof.

By “alteration” or “modulation” is meant an increase or decrease relative or compared to a suitable control. An alteration or modulation may be an increase or decrease relative to a control by as little as 0.5%, 1%, 2%, 3%, 4%, 5%, 10%, 15%, 20%, 25%, 30%, 35%, or by 40%, 50%, 60%, 70%, or even by as much as 75%, 80%, 85%, 90%, 95%, or 100%, as well as percentages there between.

By “ameliorate” is meant decrease, suppress, attenuate, diminish, arrest, abrogate, relieve, or stabilize the presence, development, or progression of a condition, disorder, or disease.

By “administering” is meant providing one or more compositions as described herein to a patient or a subject. By way of example and without limitation, composition administration, can be performed by topical application, transdermal administration, intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be oral, rectal, intravaginal, by application to skin, transdermal, by patch, or other transdermal device as known and used by those in the art.

An “analgesic component” refers to a painkiller, which may be an active ingredient or component, or any member of a group of drugs or compounds used to achieve analgesia, or relief from pain. Analgesic drugs or compounds act in various ways on the peripheral and central nervous systems. They are typically distinct from anesthetics, which temporarily affect, and in some instances, completely eliminate, sensation.

A “balm” refers to a salve or ointment composition, preparation, or product, which is typically administered or delivered topically to the skin. In an embodiment, the delivery is transdermal delivery. In an embodiment, the balm or salve comprises a fragrance, which may be imparted by natural, organic, or artificial ingredients, or combinations thereof. In an embodiment, the balm or salve treats, heals, or soothes the skin, and/or treats a neuropathic condition or disorder.

Cannabis (cannabis products) in the products, compositions and formulations described herein may be in the form of one or more cannabinoids. In some embodiments, the cannabinoid is a cannabinol, e.g., tetrahydrocannabinol (THC). In some embodiments, the cannabinoid is a cannabidiol, e.g., CBD. In an embodiment, the composition may comprise mixtures of two or more cannabinoids and other ingredients. In an embodiment, the composition may comprise mixtures of two or more cannabinoids, two or more terpenes and other ingredients. In embodiments, the other ingredients may comprise scents, oils, cannabis extracts, buffering agents, humectants, stabilizers, moisturizers, antioxidants, preservatives, and other components. In embodiments, the ingredients are natural, artificial, or a combination thereof. Illustratively, in an embodiment, cannabis ratios may include THC and 1:1 CBD:THC. In another embodiment, cannabis ratios may include 5:1 or 1:3 CBD:THC. Without limitation, such additional ingredients, e.g., scents, oils, cannabis extracts, buffering agents, humectants, stabilizers, moisturizers, antioxidants, preservatives, may be present in the products, compositions and formulations described herein in amounts of between about 0.5% to 85%, or between about 0.5% to 50%, or between about 0.5% to about 25%, or between about 0.5% and 15%, or between about 0.5% and 10%, or between about 0.2% and 5%, or between about 1% and 25%, or between about 1% and 15%, or between about 1% and 10%, or between about 1% and 5%, or between about 0.5% and 1.5%, inclusive of the first and last values.

Cannabis extracts, also called cannabis concentrates, are significantly more potent than standard cannabis buds Their applications as medicine have been effective for patients suffering from a variety of ailments. A cannabis extract may reflect the cannabis plant strain from which it was extracted; the smell, taste, and effects are simply magnified due to an increased concentration by weight. Those skilled in the art will know how to prepare cannabis extracts using processes and methods available and practiced in the art. Representative types of cannabis extracts include, by way of example and without limitation, kief, dry sieve (dry sift), hash, butane hash oil (BHO), supercritical CO₂ oil, Rick Simpson Oil (RSO) and rosin.

Kief is composed of the trichomes (the crystalline structures coating the outside surface of the cannabis flowers) enriched from the dried plant material, usually via specialized filtering screens. Kief is generally considered a lower-quality extract, but some extractors can produce an extremely clean and flavorful product using the dry sieve method. The THC content in kief can range from about 20% to 60%.

Dry sieve (sometimes referred to as “dry sift”) is a popular form of non-solvent hash. Dry sieve is a more refined version of kief that is processed through a series of filters so that only the trichome heads remain and plant matter is removed. The quality of dry sieve is often determined by the amount of plant matter and capitulate trichome stalks that remain in the final product. Optimally, the dry sieve process yields only the largest and flawless trichome gland heads, in the absence of the gland stems, plant matter, etc. that may contaminate the kief extractions. Highly purified dry sieve hash should melt completely when exposed to heat, known as full-melt dry sieve hash.

A number of processes may be used to produce hash from the cannabis plant. For example, ice water extraction is a commonly used process for generating quality, non-solvent hash. The ice water extraction process allows for the isolation or enrichment of the trichome heads from the stalks and plant matter of the cannabis plant. The trichome heads contain the essential oils of cannabis. The quality of the resulting hash is often determined by the size of the isolated trichome heads and the extent to which it melts when heated, with full-melt being optimal. Following the ice water extraction process, the final product must be fully dried to avoid the development of mold and other microbe contaminants, which are potentially harmful. In addition, the powdery kief that coats cannabis flowers can be collected and pressed together to form hash. In addition, solvents, e.g., ice water or ethanol, may be used to remove cannabinoid-loaded trichomes from a cannabis plant. Although it is not as potent as Butane Hash Oil (BHO) and other cannabis concentrates, hash is widely used because of the clean and natural extraction processes that generate it.

Butane Hash Oil (BHO) is a type of cannabis concentrate that is produced using butane as the main solvent. A number of variables, e.g., temperature, can determine the final consistency of BHO. The different consistencies of BHO are referred to by different terms: “shatter” refers to the glass-like consistency that often snaps or “shatters” when handled; and “budder, honeycomb, crumble, and sap” are terms used to describe the different textures of BHO. Under BHO extraction, the THC content can be as high as 80-90%. Accordingly, many patients suffering from chronic pain, sleep disorders, and other intractable symptoms may opt for BHO as a source of cannabis, providing that the BHO product is pure and free from contaminants, such as traces of butane or pesticides.

A carbon dioxide (CO₂) extraction process allows cannabis compounds to be extracted with low toxicity and involves the use of a high-pressure vessel containing cannabis. Supercritical CO₂ is added into the vessel and pumped through a filter where it is separated from the plant matter once the pressure is released. Thereafter, the supercritical CO₂ evaporates and is dissolved into the cannabinoids. Because CO₂ is a supercritical fluid, it converts into a liquid form when pressurized. CO₂ is also a pure chemical substance that occurs naturally and leaves behind no residues.

Whole-plant cannabis oil can be orally administered or applied directly to the skin. Such oils are also known as cannabis oil, hemp oil, Phoenix Tears, and Rick Simpson Oil (RSO). Oil forms of cannabis, e.g., tinctures (drops) afford a convenient mode of oral delivery, and intake through the oral mucosal membranes in the mouth provides for rapid and effective absorption directly into the systemic circulation because of the increased bioavailability of the cannabinoids. Similarly, application onto the skin or absorption into the skin may be achieved by topical or transdermal administration.

Whole-plant cannabis oil differs from “hemp seed oil,” which is a cold-pressed oil produced from the seeds of the hemp plant. Hemp seed oil contains essential fatty oils and is used primarily, but not solely, for its nutritional benefits. By contrast, whole-plant oil derived from the cannabis plant is produced from the buds/flower of the female cannabis plant and is comprised of many different cannabinoids, including THC, CBD, CBN, CBG, and more, in addition to terpenes and other compounds. Rick Simpson Oil often contains an increased amount of THC; other oils contain only non-psychoactive compounds like CBD.

Rosin is a solid form of resin that is obtained from cannabis by adding pressure and heat to vaporize the volatile liquid terpenes, typically using an industrial heat press. The rosin technique is efficient and cost-effective and can rapidly provide a solvent-free hash of high quality.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “disease” is meant any condition or disorder that damages or interferes with the normal function of a cell, tissue, or organ. Examples of diseases include skin conditions or disorders, such as acne, inflammatory acne vulgaris, eczema, hyperpigmentation, shingles, wrinkling, sunburn, dermatitis, as well as neurological and inflammatory conditions that cause pain and physical discomfort to an afflicted individual.

By “effective amount” is meant the amount of a required to ameliorate the symptoms of a disease or condition relative to an untreated patient. The effective amount of active compound(s) used in the compositions and methods for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. A physiologically effective amount is that which can be administered to a subject (without adverse effects) and is compatible with the subject's normal functioning and internal body systems following administration.

The terms “isolated,” “purified,” or “biologically pure” refer to material or an active ingredient that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a component or ingredient is purified if it is substantially free of chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein or active ingredient that can be subjected to modifications, for example, phosphorylation or glycosylation, different modifications may give rise to different isolated proteins or ingredients, which can be separately purified.

As used herein, “obtaining” as in “obtaining an agent” includes synthesizing, purchasing, isolating, purifying, or otherwise acquiring the agent.

The term “pharmaceutically-acceptable excipient, vehicle, carrier, or diluent” as used herein means one or more compatible solid or liquid fillers, diluents, ingredients, substances, or encapsulating substances that are suitable for administration into a human. The term “pharmaceutical composition,” as used herein, refers to a composition formulated for pharmaceutical use or to achieve a therapeutic effect. In some embodiments, the pharmaceutical composition further comprises a pharmaceutically acceptable excipient, carrier, or diluent. In some embodiments, the pharmaceutical composition comprises additional agents, e.g., for specific delivery, increasing half-life, or other therapeutic compounds. In an embodiment, a pharmaceutically acceptable substance is also physiologically acceptable in a subject.

By “reduces” is meant a negative alteration of at least 5%, 10%, 25%, 50%, 75%, 90%, 100%, or percentages therebetween.

By “reference” is meant a standard or control condition.

By “subject” is meant a mammal, including, but not limited to, a human, a non-human primate, or a non-human mammal, such as a bovine, equine, canine, ovine, or feline. A subject also refers to a patient or an individual as used herein. A subject (or patient or individual) refers to an animal (mammal) which is the object of, or candidate for, treatment, observation, assessment, or experiment. The terms “subject,” “patient,” and “individual” are used interchangeably herein. In preferred embodiments, the subject is a human being. Both male and female subjects are embraced by the described methods. A “subject in need thereof” or “patient in need thereof” refers to an individual having, diagnosed with, at risk of having, or suspected of having a condition, disease or disorder, for example, a skin condition or other condition, such as a neurological condition described herein. In particular embodiments, the subject or patient is suffering from or is at risk of pain and/or discomfort associated with a disease, disorder, condition, or pathology. In embodiments, and without limitation, the disease or disorder is selected from arthritis, Rheumatoid arthritis, diabetic neuropathy, sciatica, Ankylosing Spondylitis, or Neuromyelitis Optica Spectrum Disorder.

Ranges used herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “terpene” is generally meant any of various organic compounds, e.g., isomeric hydrocarbons (C₁₀H₁₆), produced by a variety of plants, particularly conifers, and present in essential oils, resins and balsams of the plants. Terpenes used especially as solvents and in organic synthesis. Terpenoids (or isoprenoids) are modified terpenes, which contain additional, particularly, oxygen-containing, functional groups.

“Topical administration” refers to application of a composition, formulation, or product to the skin, for example, by rubbing or massaging onto the skin, e.g., the surface of the skin.

“Transdermal administration” refers to topical application of a composition, formulation, or product onto the skin, and penetration or permeation of the composition, formulation, or product (or one or more active component(s) thereof) through the skin layer (e.g., the stratum corneum) and skin layers or circulation beneath, such that their effects are exerted on deeper or more distant tissues of the body, for example as described in M. R. Prausnitz and R. Langer, 2008, “Transdermal drug delivery,” Nature Biotechnology, 26(11):1261-1268; or R. L. Wilbur, 2017, White paper, Gensco Pharma. In some cases, transdermal products utilize methods of enhancing penetration through the stratum corneum, the primary barrier of the skin, allowing sufficient amounts of a product or active to either reach the systemic circulation or deeper underlying tissues. While topically applied compounds may only minimally penetrate the skin layer, transdermal administration affords a site specific treatment, eliminating or minimizing issues with co-morbidities, adverse drug reactions, drug-drug interactions, and side effects, that may have adverse effects. In some cases, topically applied compositions, formulations, or products, and/or (an) active component(s) thereof, may penetrate the outer layer of the skin following application by rubbing or massaging on the skin and target sites within the skin or below it, as well as target and reach more distant sites beyond the local site of application (e.g., systemic treatment).

In general, topical application of a composition, formulation, or product (e.g., a medication) to the skin relies on passive diffusion into the skin itself, thus creating a more local effect. For transdermal administration, a composition, formulation, or product (e.g., a medication) is applied to the skin and involves skin penetration of the composition, formulation, or product (or active(s) thereof), which cross the outer skin barrier, enter the systemic circulation, and exert effects in areas other than the site of application. In some cases, enhancing compounds, ingredients, or technology that increase the amount of a composition or active that can cross the skin barrier may be employed. (R. L. Wilbur, 2017, White paper, Gensco Pharma).

Without intending to be bound or limited by scientific theory, and by way of example, the skin is the largest organ of the human body and comprises roughly 10% of the body's mass. The primary function of the skin is as a barrier between the body and the external environment. As a barrier, the skin protects against UV radiation, infection by or entry of microorganisms, allergens, and chemicals, and loss of water and nutrients. In addition, the skin is involved in thermal regulation, metabolism, and blood pressure control. As a sensory organ, the skin provides environmental information, e.g., temperature, pressure, and noxious stimulation, such as pain. Human skin is composed of three main regions: the epidermis, dermis, and subcutaneous tissues. Appendages associated with the skin include hair follicles and eccrine and apocrine sweat glands. The outer most layer of the epidermis, the stratum corneum (horny layer) of the skin, provides the main barrier and is composed of 10-20 μm of high density, low hydration cell layers about 10-15 cells deep. Corneocytes of the stratum corneum act as “bricks” in a matrix of intercellular lipids that are structurally arranged in multiple lamellar layers within a continuous lipid domain to provide the barrier function of the stratum corneum. A permeant or penetrating ingredient, component, or molecule applied to the skin has three potential routes or pathways across the epidermis: through the appendages (e.g., hair follicles or sweat ducts), through the corneocytes (e.g., a transcellular route), through the matrix layers (e.g., an intercellular route), or a combination of any of these routes. Intercellular lipid bilayers occupy only a small area of the stratum corneum; however, they provide a continuous path through the stratum corneum, which may serve as a route for permeation of components, drugs, or actives through the stratum corneum. Both lipid and polar molecules may be transported through the intercellular route, and the amount and rate of diffusion depend upon the physiochemical properties of a permeant (e.g., an ingredient, drug, or active component) in a vehicle or composition.

Without intending to be bound or limited by scientific theory, the permeation process generally involves the release of the permeant from the vehicle or composition, followed by the diffusion into and through the stratum corneum, then partitioning into the more aqueous epidermal environment and diffusion into deeper tissues or uptake by the cutaneous circulation. These processes are highly dependent on the molecular size, physicochemical properties, solubility and diffusivity of the permeant within each environment. Nonlimiting strategies that have been developed to facilitate drug permeation through the epidermis include physical enhancement methods (e.g., micro needles, sonophoresis, iontophoresis, micro abrasions), which actively disrupt the skin structure; passive diffusion enhancement, which is achieved by increasing the thermodynamic activity of an active in formulations (supersaturations); and permeation enhancers, for example, chemical penetration enhancers (CPEs), which interact with skin constituents to promote active or drug flux.

As known and used by skilled practitioners in the art, CPEs are pharmacologically inactive compounds that diffuse into and partition the skin, and reversibly interact with the stratum corneum components, specifically the intercellular lipid bilayers, resulting in the production of pore or channels in the lipid bilayers through which active molecules can pass. Selection of CPEs is within the skill of those in the art, based upon a permeant molecule's physiochemical properties, manner of application, and risk of skin damage. Selection and formulation of one or more CPEs with a specific permeant (active or drug) can increase the transdermal absorption of the active or drug from 1-5% of active or drug applied compared with topical application with no CPE, to rates greater than 40%. Enhancement of active drug crossing the skin (e.g., flux), allows for deeper tissue penetration of the drug and uptake by the cutaneous circulation providing for systemic activity.

By way of further example, transdermal patches may be used as transdermal delivery systems for pharmaceuticals and active components in compositions and formulations. Patches can increase active or drug absorption due to prolonged application times. In addition, the formulation matrix or reservoir of the patch can maintain the active or drug concentration gradient within the device after application so as to sustain delivery of active or drug to the interface between the patch and the skin. The high active or drug concentration and occlusive nature of the patch can drive modest amounts of drug through the skin over time. Other CPE compounds and technology may also include the use of nanoparticle delivery systems, micelles, vehicles such as gels, patches, films, and the like, that can be used in combination with patches to increase transdermal penetration of a permeant to achieve a desired outcome.

It will also be understood that, in embodiments, an active ingredient or permeant, such as a cannabinoid, of a composition, formulation, or product as described herein can transdermally permeate the skin and enter the circulation beneath the stratum corneum following topical application of the composition, formulation, or product to the skin. As noted supra, transdermal permeation of an active molecule or permeant depends on its physicochemical properties, including molecular size. In the case of cannabinoids, such as THC and CBD, and the like, their size of about 312 Daltons may allow these components to more readily pass through the stratum corneum of the skin, thereby providing transdermal administration of these components and an ability to reach the circulation beneath the skin. By way of nonlimiting example, an active or component of about 500 Daltons or greater in size might have more difficulty passing through the outer skin layer unless it is in combination with a CPE or other permeation enhancing technology.

As used herein, the terms “treat,” treating,” “treatment,” and the like refer to reducing, abrogating, diminishing, relieving, abating, or ameliorating a disorder and/or symptoms associated therewith. It will be appreciated that, although not precluded, treating a disorder or condition does not require that the disorder, condition or symptoms associated therewith be completely eliminated. Treatment or treating refer to obtaining a desired pharmacologic and/or physiologic effect. In some cases, the effect is therapeutic, i.e., the effect partially or completely cures a condition or disease and/or adverse symptom attributable to the condition or disease. In some cases, the effect is preventative, i.e., the effect prevents an occurrence or reoccurrence of a condition or disease. To this end, the described methods comprise administering a therapeutically effective amount of the compositions as described herein.

As used herein, the terms “prevent,” “preventing,” “prevention,” “prophylactic treatment” and the like refer to reducing the risk or probability of developing a disorder or condition in a subject, who does not have, but is at risk of or susceptible to, developing a disorder or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided and described herein.

### BRIEF DESCRIPTION OF THE DRAWINGS

FIG. 1 depicts the results of a test strip assay to determine the amount of THC absorbed or penetrated into the skin of an animal (pig) at 10 minutes after topical application of a composition containing THC (1000 mg) and other carrier components onto the skin. Shown in FIG. 1 are the amounts of THC (ppm) that had absorbed or penetrated into an indicated skin layer at 10 minutes following topical application of the composition, e.g., a salve composition, comprising THC onto the skin of a pig as described in Example 6.

FIG. 2 depicts the results of a test strip assay to determine the amount of THC absorbed or penetrated into the skin of an animal (pig) at 30 minutes after topical application of a composition containing THC (1000 mg) and other carrier components onto the skin. Shown in FIG. 2 are the amounts of THC (ppm) that had absorbed or penetrated into an indicated skin layer at 30 minutes following topical application of the composition, e.g., a salve composition, comprising THC onto the skin of a pig as described in Example 6.

FIG. 3 depicts the results of a test strip assay to determine the amount of THC absorbed or penetrated into the skin of an animal (pig) at 60 minutes after topical application of a composition containing THC (1000 mg) and other carrier components onto the skin. Shown in FIG. 3 are the amounts of THC (ppm) that had absorbed or penetrated into an indicated skin layer at 60 minutes following topical application of the composition, e.g., a salve composition, comprising THC onto the skin of a pig as described in Example 6 herein.

### DETAILED DESCRIPTION OF THE EMBODIMENTS

Featured herein are compositions (also termed formulations) that are useful for treating and healing skin conditions and damaged skin, as well as for treating and relieving neurological symptoms and conditions, including inflammation, that result in pain and physical discomfort to an individual in need.

Also provided are methods of treating a condition, disease, disorder, or symptoms thereof, which comprise administering a therapeutically effective amount of a pharmaceutical composition to a subject (e.g., a mammal such as a human). In an embodiment, the condition, disease, or disorder is a skin condition, disease, or disorder. In an embodiment, the condition, disease, or disorder is a chronic or acute injury, e.g., a wound. In an embodiment, the condition, disease, or disorder is an inflammatory condition, disease or disorder, e.g., rheumatoid arthritis. In an embodiment, the condition, disease, or disorder is a neurological condition, disease or disorder, or one associated with pain, such as fibromyalgia, arthritis, Rheumatoid arthritis, diabetic neuropathy, sciatica, Ankylosing Spondylitis, or Neuromyelitis Optica Spectrum Disorder. In other embodiments, the condition, disease, or disorder is psoriasis, eczema, rash, insect bites, dermatitis, acne, acne vulgaris, skin wrinkling, and the like. Thus, one embodiment is a method of treating a subject suffering from, at risk of, or susceptible to one or more of the aforementioned conditions, diseases or disorders or symptoms thereof. The methods include the step of administering to the subject a therapeutically effective amount of an amount of a composition described herein sufficient to treat or abrogate the condition disease or disorder or symptom thereof, under conditions such that the disease or disorder is treated, or relief is provided to the subject in need thereof. In a particular embodiment, the composition is administered topically (e.g., topical application to the skin). In another embodiment, the composition is administered transdermally. In an embodiment, pain experienced by the subject or patient is locally reduced, ameliorated, abated, alleviated, and the like, following topical administration of a composition described herein to an area of the body, e.g., foot, neck, shoulder, torso, leg, etc. In an embodiment, pain experienced by the subject or patient is systemically reduced, ameliorated, abated, alleviated, and the like, following topical administration of a composition as described herein to an area of the body, e.g., foot, neck, shoulder, torso, leg, etc. In an embodiment, pain experienced by the subject or patient is both locally and systemically reduced, ameliorated, abated, alleviated, and the like, following topical administration of a composition as described herein to an area of the body, e.g., foot, neck, shoulder, torso, leg, etc. In an embodiment of the foregoing, a composition as described herein is transdermally administered by methods known and used in the art.

The methods herein include administering to the subject (including a subject identified as in need of such treatment) an effective amount of a composition described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods as described herein (which include prophylactic treatment) in general comprise administration of a therapeutically effective amount of the compositions described herein, such as the compositions or formulations described in Examples 1-5 herein, to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in the described cannabis-containing compositions may be implemented.

By way of example, a cannabinoid, such as THC, may be present in a product, composition, or formulation, e.g., a lotion, salve, liniment, moisturizer, or lubricant for topical application, in an amount of from about 0.05 mg to about 250 mg, from about 0.1 mg to about 200 mg, from about 0.1 mg to about 150 mg, from about 0.2 mg to about 120 mg, from about 0.1 mg to about 100 mg, from about 0.1 mg to about 50 mg, from about 0.2 mg to about 50 mg, from about 1 mg to about 120 mg, from about 1 mg to about 20 mg, from about 1 mg to about 18 mg, from about 3 mg to about 200 mg, from about 3 mg to about 117 mg. In an embodiment, the amount of cannabinoid (or other active or ingredient) in the composition is by weight concentration (e.g., w/w or % w/w). In embodiments, the cannabinoids may include THC, THCa, THCV, CBC, CBD, CBG, CBN, or a combination thereof. In certain embodiments, the product, composition, or formulation may contain THC in an amount of about 1 mg, 2 mg, 3 mg, 4 mg, 5 mg, 5 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 48 mg, 49 mg, 50 mg, 55 mg, 60 mg, 65 mg, 70 mg, 75 mg, 80 mg, 85 mg, 90 mg, 95 mg, 100 mg, 105 mg, 110 mg, 115 mg, 116 mg, 117 mg, 120 mg, 125 mg, or more. Milligram amounts in intervals between these mg values are also encompassed herein, for example, 1.1 mg, 1.2 mg, 1.3 mg, 1.4 mg, 1.5 mg, 1.6 mg, 1.7 mg, 1.8 mg, 1.9 mg, 2.1 mg, 2.2 mg, 2.3 mg, 2.4 mg, 2.5 mg, 2.6 mg, 2.7 mg, 2.8 mg, 2.9 mg, 3.1 mg, 3.2 mg, 3.3 mg, 3.4 mg, 3.5 mg, 3.6 mg, 3.7 mg, 3.8 mg, 3.9 mg, 4.1 mg, 4.2 mg, 4.3 mg, 4.4 mg, 4.5 mg, 4.6 mg, 4.7 mg, 4.8 mg, 4.9 mg, 5.1 mg, 5.2 mg, 5.3 mg, 5.4 mg, 5.5 mg, 5.6 mg, 5.7 mg, 5.8 mg, 5.9 mg, 6.1 mg, 6.2 mg, 6.3 mg, 6.4 mg, 6.5 mg, 6.6 mg, 6.7 mg, 6.8 mg, 6.9 mg, 7.1 mg, 7.2 mg, 7.3 mg, 7.4 mg, 7.5 mg, 7.6 mg, 7.7 mg, 7.8 mg, 7.9 mg, 8.1 mg, 82 mg, 8.3 mg, 84 mg, 8.43 mg, 8.5 mg, 8.6 mg, 87 mg, 8.8 mg, 89 mg, 9.1 mg, 92 mg, 9.3 mg, 9.4 mg, 9.5 mg, 9.6 mg, 9.7 mg, 9.8 mg, 9.9 mg, etc., and greater amounts.

In embodiments, the cannabinoid CBG may be present in the product, composition, or formulation in an amount of about 0.05 mg to about 10 mg, or about 0.1 mg to about 5 mg, or about 0.2 to about 3 mg. In embodiments, the cannabinoid CBD may be present in the product, composition, or formulation in an amount of about 0.05 mg to about 15 mg, or about 0.1 mg to about 10 mg, or about 0.1 mg to about 8 mg, or about 0.1 mg to about 7.5 mg. In embodiments, the cannabinoids CBC and/or CBN may be present in the product, composition, or formulation in an amount of about 0.05 mg to about 10 mg, or about 0.1 mg to about 5 mg, or about 0.1 mg to about 1 mg, or about 0.2 mg to about 2 mg, or about 0.4 mg to about 0.6 mg. Milligram amounts in intervals between these values are also encompassed herein. In embodiments, the cannabinoids THCa and/or THCV may be present in the product, composition, or formulation in an amount of about 0.05 mg to about 10 mg, or about 0.1 mg to about 5 mg, or about 0.1 mg to about 1 mg, or about 0.2 mg to about 2 mg, or about 0.2 mg to about 1 mg, or about 0.1 mg, 0.2 mg, 0.3 mg, 0.4 mg, 0.5 mg, 0.6 mg. 0.7 mg, 0.8 mg. 0.9 mg, or 1.0 mg.

### Cannabinoids and Cannabis

In various aspects, the compositions and formulations as described herein contain a cannabis product, derivative, component, extract, active constituent, or plant portion, such as a fiber, or seed and/or one or more cannabinoids. Cannabis (also called marijuana) is a tall plant having a stiff, upright stem, divided serrated leaves and glandular hairs and is used to produce hemp fiber and as a medicinal agent or drug. Cannabis encompasses different strains of plants, among which are Cannabis indica and Cannabis sativa. A cannabinoid is any of a group of chemical compounds, including cannabinol and active constituents of cannabis, that binds to and acts on cannabinoid receptors expressed on cells in the body that affect neurotransmitter release in the brain. The cannabis plant produces over 80 cannabinoids, each of which has unique pharmacologic effects. In general, cannabinoids are obtained or collected from a female Cannabis plant. The two most abundant cannabinoids include cannabidiol (CBD) and tetrahydrocannabinol (THC). The cannabinoid composition of a cannabis plant is wide ranging and varies by strain. Some cannabis strains have higher levels of CBD, while others contain more THC. Hemp is naturally higher in CBD and contains only trace amounts of THC. In an embodiment, the compositions, formulations and products described herein contain cannabis-derived products derived solely from the mature stalks and sterilized seeds of the industrial hemp plant.

Δ⁹-tetrahydrocannabinol (THC), is the primary psychoactive compound of cannabis. Cannabinoids as used herein refers to any cannabinoid, also defined as any ligand of the cannabinoid receptor and related compounds. Cannabinoids include phytocannabinoids (obtained from plants), which comprise different classes, e.g., cannabigerol, cannabichromene, cannabidiol or CBD (non-psychoactive), cannabinol (including tetrahydrocannabinol or THC, (e.g., Δ⁹⁻THC, Δ⁸-THC). Other cannabinoids include cannabicyclol, cannabielsoin, cannabinoldiol, and cannabitriol. In some embodiments, cannabinoids suitable for the compositions, formulations and products described herein include cannabinols. In an embodiment, the compositions, formulations and products described herein include tetrahydrocannabinols, tetrahydrocannabinol (THC). A potent, naturally occurring THC stereoisomer includes Δ⁹-THC, in which the two chiral centers at C-6a and C-10a are in the trans configuration, forming the (−)-trans-isomer, a stereoisomer also known as dronobinol. There are seven double bond isomers in the partially-saturated carbocylic ring, including Δ^{6a,7}-tetrahydrocannabinol, Δ⁷-tetrahydrocannabinol, Δ⁸-tetrahydrocannabinol, Δ^{9,11}-tetrahydrocannabinol, Δ¹⁰-tetrahydrocannabinol, and Δ^{6a,10a}-tetrahydrocannabinol, according to the dibenzopyran numbering, e.g., as presented in U.S. Pat. No. 9,375,417 or 10,028,904.

By way of nonlimiting example, tetrahydrocannabinol, such as Δ⁹-THC, aids in reducing nausea and vomiting; in countering lack of appetite, e.g., in cancer and HIV/AIDS patients, respectively; and in providing relief from glaucoma. In embodiments, THC is derived from Cannabis indica or Cannabis sativa.

By way of further example, another cannabinol that may be used in the products, compositions, and formulations described herein includes tetrahydrocannabivarin (THCV), which has a propyl side chain and is psychoactive. Similar to THC, THCV is an antagonist to the CB1 and CB2 receptors expressed by cells in the body. THCV functions as an appetite suppressant and has anti-convulsive properties. Another cannabinoid that may be used in the products, compositions, and formulations described herein includes cannabinol (CBN), which provides anti-epileptic, anti-spasmodic effects and relieves intra-ocular pressure. Yet another useful cannabinoid includes (−)-trans-cannabidiol (CBD), a naturally occurring cannabidiol. CBD is found in up to 40% of the cannabinoid extracts from cannabis. Prior to processing the cannabis plant, CBD typically occurs as cannabidiolic acid (CBDa), which has a carboxylic acid at position R1. The 2-carboxylic acids of the cannabinoids can be decarboxylated to their respective decarboxylated compounds by exposure to heat, light, or alkaline conditions. CBD is obtained from non-enzymatic decarboxylation of its parent molecule, CBDA.

CBD and CBDa have been reported to be effective in a number of different therapies and treatments, for example, in treating inflammation, diabetes, cancer, mood disorders, such as post-traumatic stress disorder (PTSD) and attention deficit disorder (ADD) and neurodegenerative diseases such as Alzheimer's disease. In addition, these cannabinoids have been shown to be effective in anti-convulsive, anti-anxiety, anti-psychotic, anti-nausea and anti-rheumatoid arthritis treatments, as well as in sedation. CBD does not display the psychoactive effects of Δ⁹-THC and was found in one study to be more effective than aspirin in relieving pain and reducing inflammation. CBD has been shown to be a potent antioxidant as well as having neuroprotective and anti-inflammatory effects.

Other cannabinoids that are suitable for use in the products, compositions and formulations described herein include those of the cannabichromene (CBC). Similar to THC and CBD, CBC is derived from CBCa. CBC has been shown to inhibit the growth of cancerous tumors due to its interaction with anadamide, a human endocannabinoid. CBC is also an inhibitor of inflammation and pain, and may be used in the treatment of migraines and in stimulating bone growth. CBC is less abundant than other cannabinoids in the cannabis plant; therefore, it may be used in conjunction with CBD and THC.

Suitable cannabinoids further include those that contain a carboxylic acid substituent, also known as cannabinoid acids, such as tetrahydrocannabinolic acid (THCa) which has a carboxylic acid at R² of the cannabinoid chemical structure. The cannabinoids having a carboxylic acid substituent group are designated as “a”. By way of example, CBDa is the form of CBD that is present in a cannabis plant. The 2-carboxylic acids of the cannabinoids can be decarboxylated by exposure to heat, light, or alkaline conditions, thereby generating the respective decarboxylated compounds, such as Δ⁹-THC.

Over time, cannabinoid acids may be decarboxylated to form the corresponding phenols, such as by exposure to heat or alkaline conditions. By way of example, heating a cannabinoid acid for 5 minutes at 200-210° C. produces a decarboxylated compound. In an embodiment, a non-activated, non-psychotropic acid form of THC is THCa, which has anti-inflammatory properties and behaves similarly to THC without psychotropic side effects. THCa functions as both an anti-proliferative agent, e.g., to inhibit cell growth and proliferation, and as an anti-spasmodic agent, e.g., to quell muscle spasms, cramps and the like.

In an embodiment, cannabinoids are included in the products, compositions, and formulations as pharmaceutically acceptable salts. In embodiments, any of the abovementioned cannabinoids, alone or in combination, may be included in the products, compositions and formulations described herein. In an embodiment, a product, composition, or formulation includes the cannabinoid THC, THCa, THCV, CBC, CBD, CBDa, CBG, CBN, or combinations or mixtures thereof. In a particular embodiment, the product, composition, or formulation includes a combination of THC (or THCa or THCV), CBD (or CBDa) and CBG. In a particular embodiment, the product, composition, or formulation includes a combination of THC (or THCa or THCV) and CBG. In a particular embodiment, the product, composition, or formulation includes a combination of THC, THCa, THCV, CBN, CBC, and CBG. In a particular embodiment, the product, composition, or formulation includes a combination of THC (or THCa or THCV), CBG and CBN. Mixtures of THC (or THCa or THCV), CBD (or CBDa) and CBG may be present in a composition, formulation or product in different ratios, for example, without limitation, 1:1 w/w, 1:2 w/w; 1:3 w/w, 1:4 w/w, 1:5 w/w, 1:6 w/w, 1:7 w/w, 1:8 w/w. 1:9 w/w, 1:10 w/w, etc. or any other mixture ratio. A number of different ratios of the cannabinoids described herein can be used for the applications described herein, for example, topical and/or transdermal applications. As will be appreciated by the skilled practitioner, the ratios of cannabinoid ingredients can be adjusted based on the desired or required pharmacological effects. By way of nonlimiting example, certain cannabinoids can be enriched and/or purified from a cannabis extract using known and practical methods and techniques, for example, by fractional distillation or by harvesting the cannabis plants using different techniques or protocols. Ratios of enriched/solated/purified cannabinoids contained in the cannabinoid-based products, compositions and formulations described herein may be adjusted, for example, such as a 1:1 w/w ratio of CBD:THC. Nonlimiting ratios of cannabinoids for use include 0.1:1, 0.2:1, 0.3:1, 0.4:1, 0.5:1, 0.6:1, 0.7:1, 0.8:1, 0.9:1, 1:1, 1:1.2, 1:1.5, 1:1.3, 1:1.5, 1:1.7, 1:2, 1:3, 1:4, 1:5, 1:6, 1:7, 1:8, 1:9, or 1:10, etc., where all ratios reflect weight/weight (w/w).

For the provision of one or more cannabinoids in a product, composition, or formulation, an appropriate strain of cannabis that is enriched in one or more desired cannabinoids can be selected. For example, a strain of cannabis can be selected that is CBDa- or THCa-dominant. In an embodiment, separation and/or extraction methods as known and practiced in the art can be used to obtain enrichment of a desired cannabinoid. Processing methods may also be employed to obtain an enriched yield of a desired cannabinoid, for example, CBN can be obtained upon longer exposure of cannabis to heat.

In embodiments, cannabinoids used in the products, compositions and formulations described herein include the carboxylic acid forms of cannabinoids, or cannabinoid acids. By way of nonlimiting example, processes that do not decarboxylate the naturally-occurring cannabinoid acids such as THCa and CBDa may be employed to obtain preparations of THCa and CBDa cannabinoid acid forms. To obtain cannabinoid acids, one skilled in the art would appreciate that methods which do not include heat and/or drying steps that can result in decarboxylation of the alkaloids (i.e., carboxylic acid forms) are preferably used to minimize or prevent decarboxylation.

In embodiments, the products, compositions and formulations described herein contain from about 0.05 milligrams (mg) to about 1000 mg of cannabinoid, or from about 0.1 mg to about 500 mg, or from about 0.5 mg to about 500 mg, or from about 1 mg to about 200 mg, or from about 10 mg to about 100 mg, or from 20 mg to about 50 mg, or from about 0.1 mg to about 50 mg, or from about 1 mg to about 20 mg, or from about 5 mg to about 15 mg of cannabinoid of various types. In other embodiments, cannabinoids are present in the products, compositions and formulations described herein in an amount of about 0.1 mg, 0.2 mg, 0.3 mg, 0.4 mg. 0.5 mg, 0.6 mg, 0.7 mg, 0.8 mg. 0.9 mg, 1 mg, 2 mg, 3 mg, 4 mg, 5 mg, 6 mg, 7 mg, 8 mg, 10 mg, 12 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 115 mg, 120 mg, 150 mg, 160 mg, 170 mg, 180 mg, 190 mg, 200 mg, or more, and mg amounts therebetween, e.g., 1.1, 1.2, 1.3, 1.4, 1.5, 1.6, 1.7, 1.8, or 1.9, and the like, for any of the aforementioned amounts.

The products, compositions, and formulations described herein may be standardized to contain the desired amount of a particular cannabinoid or combination of cannabinoids as desired. For example, in a cannabinoid preparation, the amount of cannabinoid of interest is quantified using standard laboratory assays; the amount of a cannabinoid preparation to be added to a composition is determined based on the amount required to achieve the desired final amount of the desired cannabinoid. For example, if a cannabis preparation contains 50% by weight of the desired cannabinoid, the amounts of other ingredients are adjusted to maintain the amount of the desired cannabinoid.

In general, the amount of cannabinoid, in weight percent (w %), in a composition is between about 0.01% and about 5% of the composition, between about 0.05% and about 4%, between about 0.1% and about 3.5%, between about 0.2% and about 3%, between about 0.4% and about 2%, or between about 0.6% and about 1.5%. In a nonlimiting embodiment, the amount is between about 0.8% and 1.2%, or about 1%. In other embodiments, the amount of cannabinoid is about 0.01%, about 0.05%, about 0.1%, about 0.2%, about 0.4%, about 0.6%, about 0.8%, about 0.9%, about 1%, about 1.1%, about 1.2%, about 1.4%, about 1.6%, about 1.8%, about 2%, about 3%, about 4%, about 5%, about 6%, about 8%, about 10%, about 15%, about 20%, or about 25% (w %). The remaining ingredients are adjusted to maintain the desired weight percent of a particular cannabinoid.

A cannabis preparation may be made by any method or process known and used by those skilled in the art. By way of nonlimiting example, cannabis plant matter is extracted with a solvent such as heptane, butane, hexane, isopropyl alcohol, ethanol, and liquid, dry ice, or supercritical CO₂. If cannabinoid acids (“a forms”) are desired or if decarboxylated cannabinoids are desired, extraction conditions may vary accordingly. Cold extraction methods can be used when cannabinoid acids are desired. Solvent can be removed from the extract by any method known and practiced in the art, including vacuum and/or distillation/evaporation. The resulting cannabis extract or preparation is typically in an oil form or viscous oil form. In general cannabis oil that has not been heated or exposed to heat includes cannabinoid acids or “a” forms of cannabinoids that contain a carboxylic acid substituent as noted supra.

To improve the rate at which the cannabis diffuses through the skin, e.g., the stratum corneum layer of the epidermis, additional ingredients, e.g., excipients, may be included in the products, compositions and formulations described herein. By way of example, such excipients may be a carrier agent, a permeation enhancer, an adhesive, a terpene, or combinations of any of the foregoing. As would be appreciated by the skilled practitioner, some of the aforementioned compounds may act as both carrier agents and permeation enhancers, or have pharmaceutical activity.

### Terpenes

The products, compositions and formulations as described herein may also include a terpene and/or terpenoid. Terpenes are a diverse group of organic, unsaturated hydrocarbons (derived from 5-carbon isoprene units, having the formula C₁₀H₁₆) and are produced by a wide variety of plants, especially in the essential oils of plants such as conifers and citrus trees. Terpenes not only provide a unique odor for an individual cannabis plant, they provide therapeutic benefits, similar to those of cannabinoids, e.g., THC and CBD. Terpenoids are terpenes that have been chemically modified to include functional groups including heteroatoms. While terpenes are naturally present in cannabis, they can be removed during extraction processes. Accordingly, in embodiments, one or more terpenes and/or terpenoids are either naturally present in or are added to the products, compositions, or formulations. Terpenes/terpenoids have various pharmaceutical (pharmacodynamic) effects and can be selected for their desired pharmaceutical activities.

In an embodiment, the terpene/terpenoid constitutes limonene, a colorless liquid hydrocarbon that is a cyclic terpene and a chiral molecule. The D-isomer of limonene is most common and possesses a strong smell of oranges and a bitter taste. Biological sources produce one terpene enantiomer; citrus fruit is a main commercial or industrial source and contains D-limonene ((+)-limonene), which is the (R)-enantiomer (CAS number 5989-27-5, EINECS number 227-813-5). Racemic limonene is known as dipentene (IUPAC name: 1-methyl-4-(1-methylethenyl)-cyclohexene, also known as 4-isopropenyl-1-methylcyclohexenep-Menth-1,8-diene Racemic: DL-limonene; dipentene). The principle metabolites of limonene are (+)- and (−)-trans-carveol, a product of 6-hydroxylation) and (+)- and (−)-perillyl alcohol, a product of 7-hydroxylation by CYP2C9 and CYP2C19 cytochromes in human liver microsomes.

In an embodiment, the terpene/terpenoid constitutes linalool (IUPAC name: 3,7-dimethylocta-1,6-dien-3-ol), a naturally occurring terpene alcohol found in many flowers and spice plants. Because of its scent (floral and slightly spicy), linalool is frequently used commercially. It is also known as ß-linalool, linalyl alcohol, linaloyl oxide, p-linalool, allo-ocimenol, and 3,7-dimethyl-1,6-octadien-3-ol. More than 200 species of plants produce linalool, mainly in the families Lamiaceae, Lauraceae and Rutaceae, as well as in some fungi. Linalool is a precursor in the formation of Vitamin E and has been utilized in the treatment of psychosis and anxiety, as an anti-epileptic agent, as an analgesic, and as an insecticide. Linalool is suitable for use in soaps, detergents, shampoos and lotions.

In an embodiment, the terpene/terpenoid constitutes myrcene. Myrcene, or ß-myrcene (IUPAC name: 7-methyl-3-methylene-1,6-octadiene), is an olefinic natural organic compound, which is classified as a hydrocarbon and a monoterpene. As a dimer of terpenes, myrcene is a component of the essential oil of several plants, including bay, cannabis, ylang-ylang, wild thyme, mango, parsley and hops and is a key intermediate in the production of several fragrances. The structural isomer, α-Myrcene (2-methyl-6-methylene-1,7-octadiene), is not naturally occurring. Myrcene is useful as an analgesic and as an anti-inflammatory agent.

In another embodiment, the terpene/terpenoid constitutes α-pinene (IUPAC name: (1S,5S)-2,6,6-Trimethylbicyclo[3.1.1]hept-2-ene ((−)-α-Pinene)), which is a primary monoterpene in plants (e.g., conifer, pine and orange). An alkene, α-pinene contains a reactive four-membered ring that forms a number of other terpene compounds, including D-limonene. α-pinene has utility as a bronchodilator in the treatment of asthma, as an anti-inflammatory agent; as acetylcholinesterase inhibitor, and as a broad-spectrum antibiotic. Products of α-pinene include pinonaldehyde, norpinonaldehyde, pinic acid, pinonic acid and pinalic acid. In addition, α-pinene is a major constituent in turpentine.

In another embodiment, the terpene/terpenoid constitutes ß-pinene (IUPAC name: 6,6-dimethyl-2-methylenebicyclo[3.1.1]heptane; also known as 2(10)-pinene; nopinene; and pseudopinene), which is a second isomer of pinene, in addition to α-pinene. ß-pinene, a monoterpene, is one of the most abundant compounds produced by trees and is found in cumin, lemon, pine and other plants.

In another embodiment, the terpene/terpenoid constitutes caryophyllene (IUPAC name: 4,11,11-trimethyl-8-methylene-bicyclo[7.2.0]undec-4-ene), also known as ß-caryophyllene. Caryophyllene is a natural bicyclic sesquiterpene that is a constituent of many essential oils, including clove, cannabis, rosemary and hops. Caryophyllene, which includes a cyclobutane ring, is typically mixed with isocaryophyllene and α-humulene, a ring-opened isomer. Caryophyllene contributes to the spiciness of black pepper and has been reported to act as a selective agonist of cannabinoid receptor type-2 (CB2) and to exert significant cannabi-mimetic, anti-inflammatory effects in mice. Other physiological activities associated with caryophyllene include anti-nociceptive, neuroprotective, anxiolytic, antidepressant and anti-alcoholic activities.

In another embodiment, the terpene/terpenoid constitutes citral (IUPAC name: 3,7-dimethylocta-2,6-dienal), also known as 3,7-dimethyl-2,6-octadienal or lemonal. Citral may occur as a pair or a mixture of terpenoids (double-bond isomers, E and Z) having the molecular formula C₁₀H₁₆O. The E-isomer is also known as geranial or citral A, while the Z-isomer is known as neral or citral B. Other terms include citral, geranial, neral, and geranialdehyde. Citral is present in the oils of various plants, including lemon myrtle, lemongrass, verbena, lime, lemon and orange. Geranial has a marked lemon odor, while that of neral is less intense and sweet. Uses of citral include: an agent in perfumery (for its citrus quality), a flavorant and a lemon oil additive. Citral is also a potent antimicrobial and has pheromonal effects in insects.

In another embodiment, the terpene/terpenoid constitutes humulene, also known as α-humulene or α-caryophyllene, which is a naturally occurring monocyclic sesquiterpene (C₁₅H₂₄), having an 11-membered ring with 3 isoprene units containing three non-conjugated C═C double bonds, two of which are triple-substituted and one of which is double-substituted. Identified in the essential oils of Humulus lupulus (hops), humulene is an isomer of ß-caryophyllene. The two compounds are often found together in many aromatic plants. Humulene has been reported to have anti-inflammatory activity in mammals, producing effects similar to those of dexamethasone, as well as edema reducing effects and inhibitory effects on the activities of TNF-α and interleukin-1ß (IL-1ß).

By way of nonlimiting examples, additional types of terpenes/terpenoids include menthol, eucalyptol, borneol, pulegone, sabinene, terpineol, terpinolene, camphor, and thymol. In one embodiment, an exemplary terpene/terpenoid is eucalyptol. In an embodiment, the terpenes or cannabis-derived terpenes included in a product, composition, or formulation, e.g., for topical or transdermal application or administration, comprise α-pinene, ß-myrcene, D-limonene, terpinolene, or a combination thereof.

In general, a terpene/terpenoid can be included in the products, compositions and formulations described herein in an amount of between about 0.01% and about 40%, between about 0.01% and about 20%, between about 0.01% and about 5%, between about 0.01% and about 1%. between about 0.05% and about 10%, between about 0.1% and about 5%, between about 0.1% and about 1%, between about 0.15% and about 4%, between about 0.2% and about 3%, between about 0.25% and about 2%, between about 0.3% and about 1.5%, or between about 0.4% and 1%, or about 0.5% of the product, composition, or formulation. In other cases, each terpene/terpenoid can be included in the product, composition, or formulation in amounts of between about 0.1% and about 5%, between about 0.1% and about 3%, e.g., about 1%; or about 0.01%, 0.02%, 0.03%, 0.04%, 0.05%, 0.06%, 0.07%, 0.08%, 0.09%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, 2%, 3%, 4%, 5%, 6%, 7%, 8%, 9%, 10%, or greater. The weight percent of a terpene/terpenoid included in a product, composition, or formulation also depends on the weight percent of the desired cannabinoid in the cannabis preparation and can be adjusted to maintain constancy of the ratio of a penetration enhancer, if utilized, to the amount of a given cannabinoid.

### Pharmaceutical Compositions and Methods of Delivery

Provided in another aspect are compositions, particularly, pharmaceutically acceptable compositions or formulations, comprising the described and exemplified ingredients and components for use as therapeutics and for the treatment of a condition or disease as described herein, such as a skin condition, disorder, damage, or other neurological condition, or a symptom or side effect thereof, particularly those that require pain relief to an individual. In a particular embodiment, a pharmaceutical composition is provided in the form of a liniment, for example, as described in Example 1 herein. In another particular embodiment, a pharmaceutical composition is provided in the form of a salve, for example, as described in Examples 2 and 4 herein. In other embodiments, a pharmaceutical composition is provided as a lotion, for example, as described in Example 3 herein, or as a moisturizer, for example, as described in Example 5 herein.

The described compositions may contain a permeation enhancer, such as isopropyl myristate, for transdermal delivery or administration. (See, e.g., review by V. Methur et al., 2010, Asian J. Pharmaceutics, doi: 10.4103/0973-8398.72115). Without limitation, a permeation enhancer may be present in the compositions described herein in an amount of between about 1% to 50%, or between about 1% to 40%, or between about 1% to 40%, or between about 2% to 35%, or between about 2% to 25%, or between about 5% to 45% of the composition (w/w). In some embodiments, a terpene, especially a natural terpene, is included in the composition, which may be palmarosa, menthol, or myrcene. As will be appreciated by the skilled practitioner, terpenes are a diverse group of organic hydrocarbons derived from 5-carbon isoprene units and are produced by a wide variety of plants. Terpenoids are terpenes which have been chemically modified to add functional groups including heteroatoms.

The compositions described herein comprise a pharmaceutically-acceptable diluent, carrier, or excipient. In an embodiment, the pharmaceutically acceptable compositions are in unit dosage form. Conventional pharmaceutical practice may be employed to provide suitable formulations or compositions to administer the admixed components to patients suffering from a condition or disease (e.g., a skin condition or disease or a neuropathology). Administration may also begin before a patient is symptomatic. The compositions as described herein containing excipients can be prepared by any technique routinely or typically used by the practitioner in pharmacy, pharmaceutics, drug delivery, pharmacokinetics, medicine, or other related discipline in which an excipient is admixed with other components or reagents, e.g., a drug, medicament, compound, or therapeutic agent. In an embodiment, the compositions comprise one or more cannabinoids and one or more terpenes. In an embodiment, the one or more terpenes are cannabis-derived terpenes. In an embodiment, the one or more cannabinoids comprise THC, THCa, THCV, CBG, CBC, CBN, CBD, or a combination thereof. In embodiments, the cannabis-derived terpenes include α-pinene, ß-myrcene, D-limonene, terpinolene, or a combination thereof.

Any appropriate route of administration may be employed. In general, the described methods may be practiced using any mode of administration that is medically acceptable and practicable, i.e., any mode or route that produces effective levels of the active ingredients, such as cannabis, without causing clinically unacceptable, adverse effects. By way of nonlimiting example, modes and routes of administration include parenteral, intravenous, subcutaneous, intramuscular, intraorbital, ophthalmic, intracapsular, intrathecal, intracisternal, intranasal, intratracheal, aerosol, topical, transdermal, intravaginal, rectal (suppository), oral administration, or within/on implants, e.g., fibers such as collagen, or osmotic pumps, etc. Therapeutic compositions may be in the form of liquid solutions or suspensions or emulsions. By way of example, for oral administration, formulations may be in the form of a powder, tablet, pill, pellet, capsule, syrup, oil, suspension, gel, drops, or lozenge; for intranasal formulations, compositions may be in the form of powders, nasal drops, or aerosols. For topical application or administration (e.g., application on the skin epidermis), compositions may comprise oils and emollients, for formulating as lotions, gels, salves, liniments, ointments, creams, balms, unguents, and the like.

In an embodiment, the compositions as described herein may be administered or delivered to a subject by application on, in and/or around the abdomen, groin, buttocks, back, torso, neck, face, chest, legs, arms, scalp or other suitable skin surface. The compositions may be formulated such that the ingredients, such as the cannabinoid(s), are administered in patches (transdermal patches), or as ointments, emulsions, suspensions, lotions, creams, pastes, gels, sprays, foams, salves, liniments, or oils. Accordingly, via transdermal delivery methods, the cannabinoid ingredients (and other ingredients) of the compositions are delivered and made systemically available to a mammal in a therapeutically effective amount. Via topical delivery methods, the cannabinoid ingredients (and other ingredients) of the compositions provide localized benefit such as the reduction or alleviation of pain and other conditions originating near the surface of the skin.

Methods well known in the art for making formulations and compositions are found, for example, in “Remington: The Science and Practice of Pharmacy” Ed. A. R. Gennaro, Lippincourt Williams & Wilkins, Philadelphia, Pa., 2000, and updated editions thereof. Formulations suitable for administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful delivery systems for the compositions may include ethylene-vinyl acetate copolymer particles, osmotic pumps, implantable infusion systems, and liposomes. The formulations may contain excipients, for example, lactose, or may be aqueous solutions containing, for example, polyoxyethylene-9-lauryl ether, glycocholate and deoxycholate, or may be oily solutions for administration in the form of nasal drops, or for topical, epidermal, or transdermal application, e.g., as a gel, salve, liniment, lotion, ointment, cream, balm, unguent, and the like. In embodiments, the formulations or compositions comprise the active and inactive ingredients and components, for example, as described in Examples 1-5 herein. In embodiments, the excipients may include, without limitation, pharmaceutically acceptable components such as solvents, thickening agents, wetting agents, skin penetration or permeation enhancers, lubricants, emollients, and fragrance.

The compositions and formulations can be administered to human patients in therapeutically effective amounts (e.g., amounts which prevent, eliminate, or reduce a condition or disease or disorder, e.g., of the skin) to provide therapy and treatment for the disease or condition. The compositions and formulations may contain, without limitation, one or more actives, carriers, excipients binders, colorants, fragrance ingredients, preservatives, buffers, diluents, etc. The preferred dosage or administered amount of a composition as described herein is likely to depend on such variables as the type and extent of the disease or disorder, the overall health status and condition of the particular patient, the formulation of the excipients, and the route of administration. In the event that a response in a subject is insufficient at the initial doses or amounts administered, higher doses (or effectively higher doses by a different, more localized delivery route) may be employed to the extent that patient tolerance permits. Multiple doses per day are contemplated to achieve appropriate systemic levels of the active ingredients, including cannabis.

### Kits or Articles of Manufacture

Kits or articles of manufacture which include a composition as described herein are provided. Optionally, the kit includes directions for administering or delivering the compositions to a subject. In other embodiments, the kit comprises a sterile container which contains the composition; such containers can be boxes, ampules, bottles, vials, tubes, bags, pouches, blister-packs, or other suitable container forms known in the art. Such containers can be made of plastic, glass, laminated paper, metal foil, or other materials suitable for holding the composition. In an embodiment, the kit contains the composition in the form of a transdermal patch. The instructions, if provided, will generally include information about the use of the composition. In other embodiments, the instructions include at least one of the following: description of the compositions and components; methods for using the enclosed materials for the treatment of a disease; precautions; warnings; indications; clinical or research studies; and/or references. The instructions may be printed directly on the container (when present), or as a label applied to the container, or as a separate sheet, pamphlet, card, or folder supplied in or with the container.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### EXAMPLES

The following examples are provided for illustrative purposes only and are not intended to limit the scope of the invention.

### Example 1

Described herein is a formulation or composition for treating a skin condition, damaged skin, neurological symptoms, as well as pain associated therewith. In an embodiment, the formulation or composition is topically administered or applied to the skin surface or epidermis. In an embodiment, the composition is transdermally delivered, e.g., via a transdermal patch. In an embodiment, the formulation or composition may be in the form of a liniment, which is a liquid or lotion for topical application, to relieve pain. In an embodiment, the liniment comprises an oil base.

The formulation or composition as described in this Example comprises a combination of active ingredients, including one or more cannabis components. The formulation or composition is useful in the treatment and/or relief of skin conditions and disorders, such as psoriasis, acne and eczema, wrinkling, aging, wound healing, ulcers, chronic and acute injuries and inflammatory conditions or diseases, such as arthritis, e.g., rheumatoid arthritis, dermatitis. The formulation or composition is particularly suitable for relieving pain and physical discomfort associated with skin or dermatological conditions.

The formulation or composition provides a pain-relieving remedy to heal damaged or injured skin. By way of example, components of the formulation or composition, such as hydrolyzed oats, sesame oil, and antioxidants treat and provide relief for skin conditions, arthritis, and inflammation, as well as pain and discomfort associated therewith.

Table 1 below provides ingredients of a formulation or composition for skin treatment and pain relief as described herein. The active ingredients and components and their purposes and effects in the formulation or composition are presented in Table 1.

TABLE 1
Cannabis Ratios in the formulation/composition
THC and CBD, e.g., 1:1 CBD:THC

| Active | Active | |
|---|---|---|
| Ingredients | Component | Purpose |
| Sesame Oil | Essential fatty | Prevents bacterial infections, repairing damaged |
| | acids, minerals, | skin cells, anti-inflammatory, promotes homeostasis |
| | lignans | |
| Hydrolyzed Oats | Avenanthramides | Reduces swelling, stimulates cell proliferation, |
| | | improves hydration, brightens skin, improves |
| | | elasticity, anti-acne |
| D-Orientine S | Isoflavones, | Free radical scavenging properties to protect skin |
| | Ursolic Acid, | from environmental sources of aging and wrinkling |
| | Vitamin A, | |
| | Policosanols | |
| Syn Coll | Collagen | Promote the synthesis of collagen, increases skin |
| | | density |
| Aloe Vera | Vitamins, | Relief from sunburns, inflammation, and other skin |
| | minerals, sugars, | conditions. Increases cell proliferation, collagen |
| | lignin, saponins, | synthesis, and hyaluronic acid. Aids in wound |
| | salicylic acids, | healing, prevents ulcers and some dermatitis |
| | amino acids | |
| Antioxidant | Polyphenols | Inhibit oxidation that can be damaging to skin cells, |
| Complex T5 | | anti-carcinogen, anti-inflammatory |
| Vitapherol T-50 | Vitamin E | Antioxidant, moisturizer, preservative |
| Niacinamide | Vitamin B3 | Treats eczema and inflammatory acne vulgaris, |
| | | treats hyperpigmentation, reduces pore size |
| Palmarosa | Terpenes | Treat dry skin, eczema and psoriasis, anti-bacterial |
| Grapefruit | Polyphenols, | Prevents poor circulation, allergies, cellulite, fluid |
| | flavonoids, | retention, acne, and oily skin, antioxidant, anti-aging |
| | terpenes | |
| Cannabis | CBD, THC, | Relief - from pain or anxiety or neurological |
| | terpenes | symptoms and the like |

| | |
|---|---|
| Carrier | Other Ingredients |

| | | |
|---|---|---|
| Isopropyl Palmitate | Distilled Water | Ceteryl Alcohol |
| Soy Lecithin | Shea Nut Butter | Optiphen ND (Phenoxyethanol, |
| | | Benzoic acid, Dehydroacetic Acid) |
| Oleic Acid | Jojoba Oil | Glyceryl Stearate |

An example of a liniment or lotion formulation or composition, especially for topical application and dermatological use, is as described below. In an embodiment, the liniment or lotion formulation or composition is topically administered or applied to the skin surface or epidermis. In an embodiment, the composition is transdermally administered. In an embodiment, the liniment or lotion composition treats a neurological condition or disorder.

Liniment or Lotion Composition -- THC

| Ingredient | % Weight |
|---|---|
| Distilled Water | 75-85% |
| | (e.g.,79.1944) |
| Cetearyl Alcohol/ | 1-6% |
| Ceteareth-20 | (e.g., 4.1318) |
| Hydrolyzed Oats | 0.5-5% |
| | (e.g., 2.0669) |
| Shea Nut Butter | 0.5-3% |
| | (e.g., 1.7331) |
| Sesame Seed Oil | 0.5-3% |
| | (e.g., 1.7331) |
| Jojoba Oil | 0.5-3% |
| | (e.g., 1.7331) |
| Olivem 1000 (Cetearyl | 0.5-3% |
| Olivate and Sorbitan Olivate) | (e.g., 1.3769) |
| D'Orientine S (Caprylic/Capric | 0.5-3% |
| Triglycerides and Phoenix | (e.g., 1.0332) |
| Dactylifera (Date) Seed Extract) | |
| Syn-Coll (Palmitoyl Tripeptide-5 | 0.5-3% |
| and Glycerin) | (e.g., 1.0332) |
| Aloe Vera Liquid | 0.3-2% |
| | (e.g., 0.8603) |
| Optiphen ND (Phenoxyethanol, | 0.3-2% |
| Benzoic Acid, Dehyrdroacetic Acid) | (e.g., 0.8124) |
| Oleic Acid | 0.3-2% |
| | (e.g., 0.6885) |
| Sugar Mulse (Ceteryl Alcohol | 0.3-2% |
| and Cetearyl Glucoside) | (e.g., 0.6885) |
| Antioxidant Complex T5 (Gingko | 0.3-2% |
| Biloba Leaf Extract, Olive Leaf | (e.g., 0.6885) |
| Extract, Rooibos Leafb Extract, | |
| White Tea Leaf Extract, Green | |
| Tea Leaf Extract) | |
| Glyceryl Stearate | 0.2-2% |
| | (e.g., 0.5166) |
| Tocopherol | 0.1-1.5% |
| | (e.g., 0.3447) |
| Niacinamide | 0.1-1.5% |
| | (e.g., 0.3447) |
| Isopropyl Palmitate | 0.1-1.5% |
| | (e.g., 0.3442) |
| Soy Lecithin | 0.1-1.5% |
| | (e.g., 0.3442) |
| Cannabis Extract | 0.1-1.5% |
| | (e.g., 0.3322) |
| Botanically Derived Terpenes | 0.001-0.05% |
| | (e.g., 0.0033) |
| Total | 100.0 |
| Cannabinoid - THC | 100-400 mg, or |
| | 180-220 mg |
| | (e.g., 200 mg +/− |
| | 20 mg) |

The above-described liniment or lotion composition comprising THC and other ingredients for topical, e.g., transdermal, delivery or application, is, by way of example, in the form of a white or cream colored semi-solid, with a viscosity of about 35-90 cP and a weight of about 56 grams(g). In an embodiment, the percent weight of cannabinoids in the composition in one container is about 0.5%-1.0%, e.g., 0.33%. In an embodiment, the liniment or lotion is administered so as to deliver about 2.0 mg cannabinoids per dose. By way of nonlimiting example, if the container is a pump container (e.g., an about 60 mL bottle with a pump and cap, filled to about 55 g-60 g), one pump from the container delivers an about 2.0 mg dose of cannabinoids. In an embodiment, the cannabinoid such as THC is present in the product in an amount of about 180 mg-220 mg per unit. In an embodiment, the composition or formulation product is stored in a sealed container and maintained at 60-80° F., optimally, avoiding high humidity, high temperature, and direct light. In an embodiment, the liniment or lotion product as described is stable for at least one year.

Another example of a liniment or lotion formulation or composition, especially for topical application and dermatological use, is as described below. In an embodiment, the liniment or lotion formulation or composition is topically administered or applied to the skin surface or epidermis. In an embodiment, the composition is transdermally administered.

Liniment or Lotion Composition - CBD:THC 1:1

| Ingredient | % Weight |
|---|---|
| Distilled Water | 75-85% |
| | (e.g., 78.9404) |
| Cetearyl Alcohol/Ceteareth-20 | 1-6% |
| | (e.g., 4.1225) |
| Hydrolyzed Oats | 0.5-5% |
| | (e.g., 2.0530) |
| Shea Nut Butter | 0.5-3% |
| | (e.g., 1.7219) |
| Sesame Seed Oil | 0.5-3% |
| | (e.g., 1.7219) |
| Jojoba Oil | 0.5-3% |
| | (e.g., 1.7219) |
| Olivem 1000 (Cetearyl Olivate | 0.5-3% |
| and Sorbitan Olivate) | (e.g., 1.3742) |
| D'Orientine S (Caprylic/Capric | 0.5-3% |
| Triglycerides and Phoenix Dactylifera | (e.g., 1.0265) |
| (Date) Seed Extract) | |
| Syn-Coll (Palmitoyl Tripeptide-5 | 0.5-3% |
| and Glycerin) | (e.g., 1.0265) |
| Aloe Vera Liquid | 0.3-2% |
| | (e.g., 0.8609) |
| Optiphen ND (Phenoxyethanol, | 0.3-2% |
| Benzoic Acid, Dehrdroacetic Acid) | (e.g., 0.8113) |
| Oleic Acid | 0.3-2% |
| | (e.g., 0.6871) |
| Sugar Mulse (Ceteryl Alcohol | 0.3-2% |
| and Cetearyl Glucoside) | 0.6788 |
| Antioxidant Complex T5 (Gingko | 0.3-2% |
| Biloba Leaf Extract, Olive Leaf | (e.g., 0.6788) |
| Extract, Rooibos Leaf Extract, | |
| White Tea Leaf Extract, Green | |
| Tea Leaf Extract) | |
| Cannabis Extract | 0.1-1.5% |
| | (e.g., 0.6623) |
| Glyceryl Stearate | 0.2-2% |
| | (e.g., 0.5133) |
| Tocopherol | 0.1-1.5% |
| | (e.g., 0.3477) |
| Niacinamide | 0.1-1.5% |
| | (e.g. 0.3477) |
| Isopropyl Palmitate | 0.1-1.5% |
| | (e.g., 0.3436) |
| Soy Lecithin | 0.1-1.5% |
| | (e.g,. 0.3436) |
| Botanically Derived Terpenes | 0.001-0.05% |
| | (e.g., 0.0066) |
| Total | 100.0 |
| Cannabinoid - CBD:THC 1:1 | 180 mg-220 mg |
| | THC; |
| | 180 mg-220 mg |
| | CBD |

The above-described liniment or lotion composition comprising cannabinoids CBD and THC (1:1) and other ingredients for topical, e.g., transdermal, delivery or application, is, by way of example, in the form of a white or cream colored semi-solid, with a viscosity of about 35-9 cP and a weight of about 56 g. In an embodiment, the percent weight of cannabinoids in the composition in one container is about 0.5%-1.5%, e.g., 0.66%. In an embodiment, the liniment or lotion is administered so as to deliver about 4.0 mg cannabinoids per dose. By way of nonlimiting example, if the container is a pump container (e.g., an about 60 mL bottle with a pump and cap, filled to about 55-60 g), one pump from the container delivers an about 4.0 mg dose of cannabinoids. In an embodiment, the cannabinoids such as CBD and THC are present in the product in an amount of about 200 mg CBD ±20 mg per bottle and about 200 mg THC ±20 mg per bottle. In an embodiment, the composition or formulation product is stored in a sealed container and maintained at 60-80° F., optimally, avoiding high humidity, high temperature, and direct light. In an embodiment, the liniment or lotion product as described is stable for at least one year.

### Example 2

Described herein is a formulation or composition, especially for topical and dermatological use. In an embodiment, the formulation or composition may be used as a salve (or balm), such as an ointment to promote healing of the skin, for example, a liquid or lotion for topical application, to relieve pain or discomfort or to afford protection, e.g., to the skin. In an embodiment, the composition is a salve. In an embodiment, the salve comprises an oil base. In an embodiment, the formulation or composition is topically administered or applied to the skin surface or epidermis. In an embodiment, the composition is transdermally delivered, e.g., via a transdermal patch or other transdermal device as used in the art.

The formulation or composition as described in this Example comprises at least one analgesic component, such as capsaicin, and at least one cannabis component as active ingredients and is useful in the treatment and/or relief of skin conditions and disorders, such as bruises, aches, sprains, acne, shingles, chronic and acute injuries and inflammatory conditions or diseases, as well as fibromyalgia and other neurological symptoms and conditions.

The formulation or composition provides a healing salve for topical application to a dermatological area. By way of example, components of the formulation or composition, such as capsicum, soothes neuropathic symptoms, while arnica and seabuckthorn reduce swelling and promote healing from skin conditions, acute injuries, and inflammation.

Table 2 below provides ingredients of the described salve formulation or composition for the treatments as described herein. The active ingredients and components and their purposes and effects in the formulation or composition are presented in Table 2.

TABLE 2
Cannabis Ratios in the formulation/composition
CBD:THC 5:1 or CBD:THC 1:3

| Active | Active | |
|---|---|---|
| Ingredients | Component | Purpose |
| Sea Buckthorn | Vitamin A, B1, | Prevent infection, slows aging process, tissue |
| Oil | B2, B6, C | regeneration, anti-neoplastic |
| Capsicum Oil | Capsaicin | Pain relief from shingles, arthritis, fibromyalgia, |
| | | migraines. Used for neuropathy, back pain, muscle |
| | | spasms, and other painful conditions |
| Arnica | Sesquiterpene | Pain and swelling relief associated with bruises, |
| | lactones | aches, sprains, insect bites, arthritis, muscle and |
| | | cartilage pain, acne, and numbness |
| Tea Tree | Terpinen-4-ol | Antibacterial, anti-inflammatory, antiviral, and |
| | and 1,8-cineole | antifungal properties. Reduces allergic skin |
| | | reactions |
| Eucalyptus | Eucalyptol | Helps with pain and inflammation, antifungal, |
| | | antibacterial, chemo-preventative |
| Calendula | Carotenoids and | Help new tissue grow in wounds, anti-inflammatory |
| | triterpenic | |
| | alcohols | |
| Cannabis | CBD, THC, | Relief - from pain or anxiety or neurological |
| | terpenes | symptoms (and the like) |
| Vitapherol T-50 | Vitamin E | Antioxidant, moisturizer, preservative |

| Carrier | Other Ingredients | |
|---|---|---|
| Isopropyl Palmitate | Shea Nut Butter - | Lanolin |
| | Virgin Organic | |
| Soy Lecithin | Beeswax | Olive Oil |
| Oleic Acid | Sunflower Oil | Optiphen ND (Phenoxyethanol, |
| | | Benzoic acid, Dehydroacetic |
| | | Acid) |

In one embodiment, the salve or balm composition comprises cannabinoids in a ratio, for example, of CBD:THC 1:3 and other ingredients for topical, e.g., transdermal, delivery or application. In an embodiment, the salve comprises from 150-350 mg CBD:THC 1:3, e.g., 250 mg CBD:THC 1:3. In an embodiment, the salve is, by way of example, in the form of an orange-colored, semi-solid balm with a weight of about 30 g. In an embodiment, the percent weight of cannabinoids in the composition in one container is about 0.0.55%-1.0%, e.g., 0.83%. In an embodiment, the total cannabinoid amount present in the salve is about 50 mg-75 mg, e.g., 56.25 mg-68.75 mg CBD per unit and about 150 mg-225 mg, e.g., 168.75 mg-206.25 mg THC per unit. By way of nonlimiting example, a container comprising the salve product contains from 62.5 mg 6.25 CBD and 187.5 mg THC 18.75 mg. In a particular embodiment, the container is a roll-on container, which contains about 20-45 mL, e.g., 30 mL, (20 g-45 g, e.g., 30 g, fill) with a cap. In an embodiment, the composition or formulation salve product is stored in a sealed container and maintained at 60-80° F., optimally, avoiding high humidity, high temperature, and direct light. In an embodiment, the salve product as described is stable for at least one year.

In another embodiment, the salve or balm composition comprises cannabinoids in a ratio, for example, of CBD:THC 5:1 and other ingredients for topical, e.g., transdermal, delivery or application. In an embodiment, the salve comprises from 150-350 mg CBD:THC 5:1, e.g., 250 mg CBD:THC 5:1. In an embodiment, the salve is, by way of example, in the form of an orange-colored, semi-solid balm with a weight of about 30 g. In an embodiment, the percent weight of cannabinoids in the composition in one container is about 0.0.55%-1.0%, e.g., 0.83%. In an embodiment, the total cannabinoid amount present in the salve is about 150 mg-250 mg, e.g., 187.47 mg-229.13 mg CBD and about 30 mg-50 mg, e.g., 37.5 mg-45.84 mg THC per unit. By way of nonlimiting example, a container comprising the salve product contains from 208.3 mg CBD 20.83 mg and 41.67 mg THC 4.17 mg. In a particular embodiment, the container is a roll-on container, which contains about 20-45 mL, e.g., 30 mL, (20 g-45 g, e.g., 30 g, fill) with a cap. In an embodiment, the composition or formulation salve product is stored in a sealed container and maintained at 60-80° F., optimally, avoiding high humidity, high temperature, and direct light. In an embodiment, the salve product as described is stable for at least one year.

Other ingredients, such as natural, organic ingredients, included in the above-described salve or balm compositions and products, for example, Shea Nut Butter, Beeswax, Sunflower Oil, Lanolin, Olive Oil, Oleic Acid, Arnica Extract, Sea Buckthorn Oil, Tea Tree Oil, Eucalyptus Oil, Isopropyl Palmitate, Soy Lecithin, Capsicum Oil, Calendula Extract, Optiphen ND (Phenoxyethanol, Benzoic Acid, Dehydroacetic Acid), Tocopherol and Activated Cannabis Extract. In a particular embodiment, other ingredients in the salve or balm, which may include natural and/or artificial ingredients and additives, are presented below.

| Ingredient | % Weight |
|---|---|
| Shea Nut Butter | (30-70%) |
| | (e.g., 50.0120) |
| Beeswax | (20-25%) |
| | (e.g., 21.1879) |
| Sunflower Oil | (5-10%) |
| | (e.g., 7.9685) |
| Lanolin | (2.5-8%) |
| | (e.g., 5.9970) |
| Olive Oil | (1-5%) |
| | (e.g., 2.8463) |
| Oleic Acid | (1-5%) |
| | (e.g., 2.0036) |
| Arnica Extract | (0.5-5%) |
| | (e.g., 1.7733) |
| Sea Buckthorn Oil | (0.5-3%) |
| | (e.g., 1.2621) |
| Tea Tree Oil | (0.5-3%) |
| | (e.g., 1.1423) |
| Eucalyptus Oil | (0.5-3%) |
| | (e.g., 1.1423) |
| Optiphen ND (Phenoxyethanol, | (0.5-3%) |
| Benzoic Acid, | (e.g., 1.1054) |
| Dehydroacetic Acid) | |
| Isopropyl Palmitate | (0.5-3%) |
| | (e.g., 1.0018) |
| Soy Lecithin | (0.5-3%) |
| | (e.g., 1.0018) |
| Cannabis Extract | 0.1-1.5% |
| | (e.g., 0.8264) |
| Capsicum Oil | 0.1-1.5% |
| | (e.g., 0.4606) |
| Calendula Extract | 0.1-1% |
| | (e.g., 0.2211) |
| Tocopherol | 0.005-0.15% |
| | (e.g., 0.0461) |
| Total | 100.00 |

### Example 3

Described herein is a formulation or composition, especially for topical and dermatological use. In an embodiment, the formulation or composition is a lotion, for topical application. In an embodiment, the lotion formulation or composition is topically administered or applied to the skin surface or epidermis.

The ingredients in the lotion described in this Example include the following: Cannabinoids: THC (105 mg-120 mg, e.g., 117.0 mg) and CBG (1.0 mg-5.0 mg, e.g., 3.0 mg). Other ingredients in the lotion include Shea butter, Sesame oil, Jojoba oil, Hydrolyzed oats, Ceteryl alcohol, D'Orientine S, Syn Coll, Oleic acid, Olivem, Aloe vera liquid, Optiphen ND, Antioxidant complex T5, Glyceryl stearate, Vitapherole T-50, Niacinamide, Isopropyl palmitate, Soy lecithin, Medical cannabis, Palmarosa and Grapefruit.

### Example 4

Described herein is a formulation or composition, especially for topical and dermatological use. In an embodiment, the formulation or composition is a salve, for topical application. In an embodiment, the salve formulation or composition is topically administered or applied to the skin surface or epidermis.

The ingredients in the salve (“Salve 1:3”) described in this Example include the following: Cannabinoids: THC (10 mg-20 mg, e.g., 17.84 mg), CBD (2 mg-10 mg, e.g., 7.44 mg) and CBG (0.5 mg-3 mg, e.g., 1.04 mg); Terpenes (cannabis derived): α-Pinene (0.005%-0.1%, e.g., 0.06%), ß-Myrcene (0.005%-0.05%, e.g., 0.02%), D-Limonene (0.005%-0.1%, e.g., 0.07%) and Terpinolene (0.005%-0.07%, e.g., 0.03%). Other ingredients in the salve include Shea nut butter, Beeswax, Sunflower oil, Lanolin, Olive oil, Seabuckthorn oil, Capsicum oil, Oleic acid, Arnica, Tea Tree, Eucalyptus, Isopropyl palmitate, Soy lecithin, Optiphen ND, Calendula, Medical cannabis and Vitapherol T-50.

### Example 5

Described herein is a formulation or composition, especially for topical and dermatological use. In an embodiment, the formulation or composition is a moisturizer or lubricant, for topical application. In an embodiment, the moisturizer or lubricant formulation or composition is topically administered or applied to the skin surface or epidermis.

The ingredients in the moisturizer described in this Example include the following: Cannabinoids: THC (23 mg-55 mg, e.g., 48.8 mg), THCa (0.05 mg-0.8 mg, e.g., 0.4 mg), CBN (0.05 mg-0.8 mg, e.g., 0.6 mg), THCV (0.05 mg-0.8 mg, e.g., 0.6 mg), CBC (0.05 mg-0.8 mg, e.g., 0.4 mg) and CBG (0.05 mg-0.8 mg, e.g., 0.2 mg). Other ingredients in the moisturizer include medium chain triglyceride (MCT) oil, Aloe vera and medical cannabis. MCT oil contains medium-length chains of triglycerides, most commonly extracted from coconut oil, or from palm oil. In embodiments, the MCT oil includes caprylic or capric acid.

### Example 6

A composition as described herein, e.g., in the form of a salve containing 1000 mg THC and carrier components was tested for its absorbance into skin following topical application to the skin of an animal (pig).

The absorbance testing procedure was performed as follows: the salve/lotion composition was applied topically to an area of the skin of a pig. Approximately 500 mg of the salve/lotion composition was applied to the area of skin using a gloved finger. The composition was applied to the skin by rubbing in a circular motion. The applied composition (salve composition) remained on the skin for different time periods, i.e., 10 minutes, 30 minutes, or 60 minutes, to allow the components of the composition, e.g., THC, to absorb into the skin. At the end of each time period, the area of the skin on which the composition had been applied was contacted with adhesive strips to test for the presence of residual cannabinoids, using an adhesive test tape (strip) method, e.g., as known in the art. (See, e.g., A. L. M. Ruela et al., 2016, BJPS, Vol. 52, No. 3:527-544; S. Zsiko et al., 2019, Sci. Pharm. (Review), Vol. 87, No. 19; doi:10.3390/scipharm87030019, 21 pages). In general, the tape-stripping technique is employed for analysis of penetration of an active or component (e.g., THC) into the stratum corneum. The procedure may be applied using an animal skin model, such as pig skin, e.g., a portion of skin removed from the animal. The procedure involved the sequence removal of cell layers of the stratum corneum by application of pieces (strips) of adhesive tape. The residues of the applied composition or formulation were removed from the skin surface, and the adhesive strip was applied by using pressure to ensure its adhesion to the skin. In particular, the tape strips were placed on the same location on the pig skin, and pressure was applied using a roller to ensure consistency in applied pressure. When the adhesive strip was removed, a portion of the stratum corneum was also removed. The first strip of tape was discarded to eliminate any residue of the composition. The succeeding strips of tape were sequentially applied to the animal's skin to strip the skin on the site where the composition was applied. The pressure and rate of application was the same during the entire procedure to assure homogeneity of the relative amount of stratum corneum that was removed. In particular, each tape strip was removed at a similar angle to ensure that a consistent volume of the stratum corneum was removed each time. The tape strips were collected and the active or component, i.e., THC, was extracted using methanol as solvent. (A. L. M. Ruela et al., 2016, BJPS, Vol. 52, No. 3:527-544).

In this Example, the test strip procedure was repeated for multiple layers of skin, e.g., 20, in order to reach the next layer of the dermis beneath the stratum corneum and assess the ability of the cannabinoid active(s) (e.g., THC, CBD), to enter the bloodstream. The stratum corneum is 10-40 μm, and in the experiments, 20 test strips were applied and pulled from the same area of the skin for each time period.

To analyze, measure, and map the absorption rate of the THC component of the composition into the skin, the test strips were dissolved in 200 μL of methanol and then were sonicated for 30 minutes at 40° C. Following sonication, the supernatant was removed and subjected to HPLC analysis.

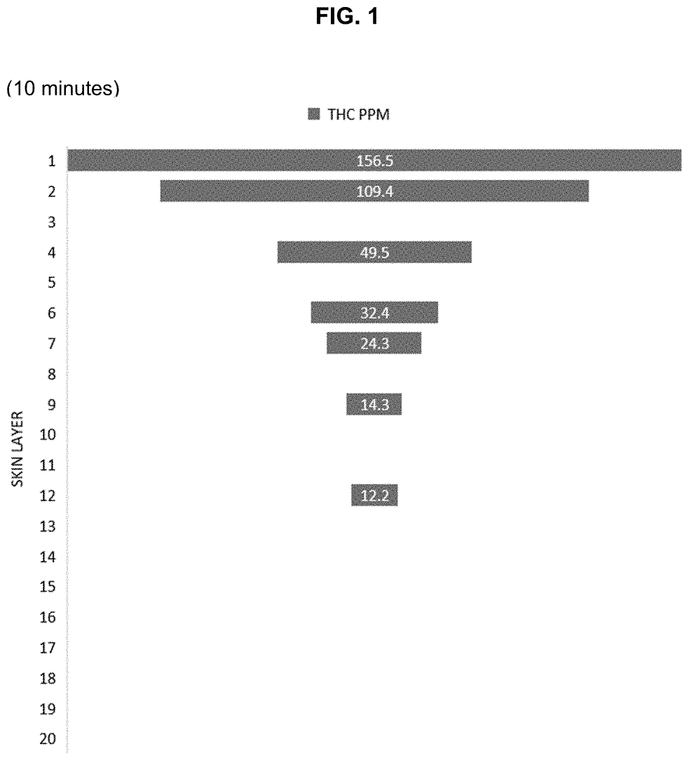

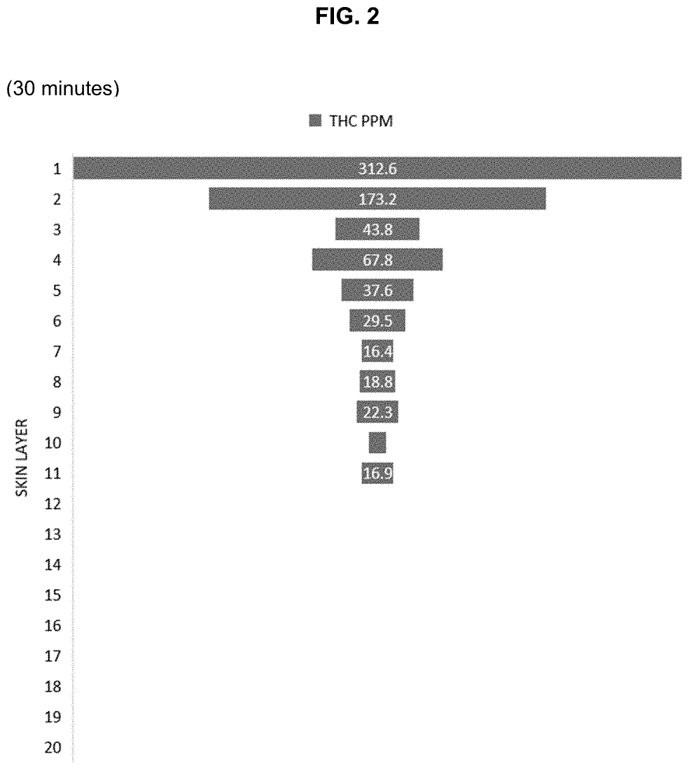

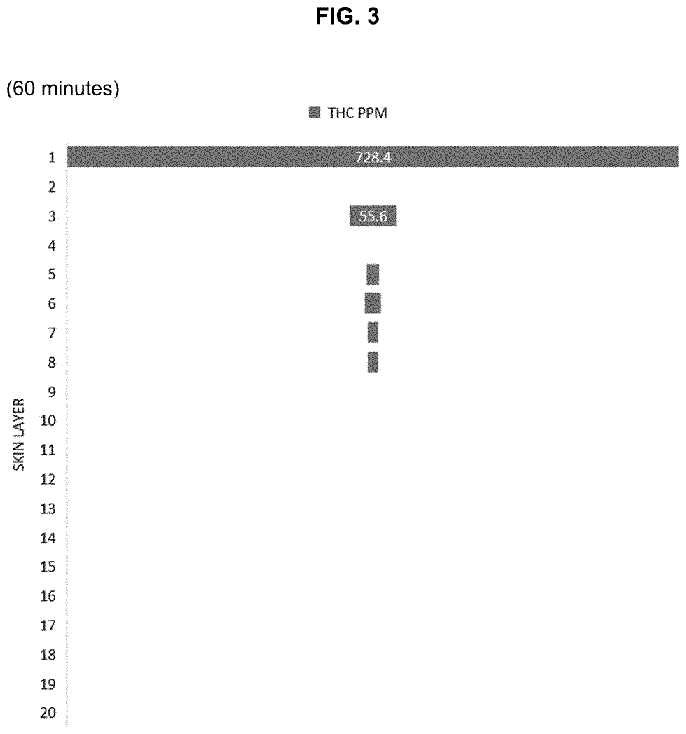

FIG. 1 illustrates the results of the time course experiment following absorption testing 10 minutes after the composition comprising 1000 mg THC+carrier ingredients was applied to the animal's skin. Absorption or permeation of THC (ppm) per layer of skin (20 skin layers deep) was assessed using adhesive test strips. FIG. 2 illustrates the results of the time course experiment following absorption testing 30 minutes after the composition comprising 1000 mg THC+carrier ingredients was applied to the animal's skin. Absorption or permeation of THC (ppm) per layer of skin (20 skin layers deep) was assessed using adhesive test strips. FIG. 3 illustrates the results of the time course experiment following absorption testing 60 minutes after the composition comprising 1000 mg THC+carrier ingredients was applied to the animal's skin. Absorption or permeation of THC (ppm) per layer of skin (approximately 20 skin layers deep) was assessed using adhesive test strips.

As determined from the experiments and observed from the results, absorption or penetration of THC of the composition into the skin was highest at 10-30 minutes after topical application to the skin. At 60 minutes after topical application, the majority of the THC component had entered the stratum corneum, the horny outer layer of the skin (epidermis). It is likely that the 5 ppm limit of quantification (LOQ) in the experiments contributed to an inability to detect the presence of cannabinoids in the skin layers beneath the stratum corneum.

### Example 7

This Example describes the effectiveness of the compositions and formulations described and exemplified supra in treating a number of different individuals (patients) for a number of different neuropathic conditions or disorders, such as pain, nerve pain, diabetic neuropathy, sciatica, pain related to Ankylosing Spondylitis, rheumatoid arthritis, and nerve damage associated with Neuromyelitis Optica Spectrum Disorder, e.g., by mitigating pain.

Patient A, who is a nurse, is on her feet for long periods of time during the day. She has arthritis and extreme nerve pain in her feet. Despite taking prescription medications for treatment, these have not alleviated or ameliorated her condition and/or the pain, which she described as feeling like walking on glass all the time. In addition, Patient A's feet always feel very tight. Patient A topically applied a salve composition as described above, i.e., a CBD 5:1 salve, to skin areas of her feet. Thirty (30) minutes following this application, Patient A felt no pain in her feet when she got up and walked across the room. This results was highly surprising, as the elimination of pain in her feet had not resulted after taking other medications, such as prescription drugs. Patient A topically applied the salve to her feet upon feeling foot pain, e.g., one or two times per day. The application of the salve to areas of her feet reduced the foot pain that Patient A experienced prior to use of the salve such that her feet were essentially pain-free from using the product. If Patient A experienced breakthrough pain, i.e., pain in an area of her foot that had not received a topical application of the salve, she could subsequently apply the salve to that spot to relieve and reduce the pain in that area of the foot as well. In addition, Patent A was extremely happy and uplifted by the results that she experienced through the use of the product.

Patient B is a man in his 70's in age and has severe arthritis in his hands. His fingers are curled up from the arthritis, and he is limited as to what he can do, e.g., limited movement and/or physical activity. Patient B topically applied a salve composition as described above, i.e., a CBD 5:1 salve, to the skin of his hands. After this topical application, e.g., about 10 minutes to about 60 minutes or longer following application, Patient B was able to use his hands and work in his garden, a task that he had not been able to do in many years. Topical application of the composition to his hands allowed Patient B to move them (partially open and close his hands in a fist) to a greater degree than prior to his use of the composition. In addition, Patient B's spirits greatly improved, and he was happier as a result of having mobility in his hands restored than he had been for many years prior to using the product.

Patient C, a man in his mid-30's in age, suffers from diabetic neuropathy in his feet. Patient C topically applied a salve composition as described above, i.e., a CBD 5:1 salve, to skin areas of his feet. After topical application of the product, e.g., about 10 minutes to about 60 minutes or longer following application, Patient C experienced notable and lasting pain relief. Patient C's use of the product allowed him to stand on his feet and to walk at least twice the distance that he was able to walk prior to his use of the product.

Patient D is an older male who has sciatica in his lower back and neck. Following topical application of a salve composition as described above, i.e., a CBD 5:1 salve, to affected areas of his lower back and his neck, e.g., about 10 minutes to about 60 minutes or longer following application, Patient D experienced reduction in pain and relief from pain, along with increased mobility. The topically applied composition was the first topical product that Patient D had used that had effectively provided him with pain relief and increased mobility.

Patient E is a female in her late 40's in age, who is suffering from Ankylosing Spondylitis and severe pain in her lower back. Patient E topically applied a salve composition as described above, i.e., a CBD 5:1 salve or a 1.3 salve, to skin areas of her lower back. Following application, e.g., about 10 minutes to about 60 minutes or longer following application, Patient E reported a noticeable decrease in pain and discomfort, as well as a reduction in pressure in her lower back. Following application of the product, Patient E reported being able to walk and stand for longer periods of time than she had been able to prior to the application of the product.

Patient F, a male in his 70's in age, suffers from Rheumatoid Arthritis. Having never previously used cannabis as a medicament and lacking success using prescription medications to treat his condition, Patient F used compositions as described supra at the recommendation of his physician. After topical application of a 1:3 salve composition to the skin of his feet, Patient F reported that he felt “really good” and pleasant, and experienced improvement in sensations, such as dullness of pain, visualization of brighter colors, and euphoria, which was followed by an increase in appetite, and then drowsiness. Of note, these improvements in pain and experiences of other sensations were felt all over Patient F's body, even though the product had been applied to his feet. Thus, components, e.g., THC, CBD active components, in the described composition applied topically to the feet were transdermally disseminated in the patient and generated a systemic effect (in his body) as well as a local effect (in his feet) by reducing pain. In addition, Patient F also topically applied a salve composition as described above, i.e., a CBD 5:1 salve, to his feet and reported much pain relief and improved mobility, but without any accompanying “head effects” sensation. Patient F was able to mitigate pain and experience pain relief from Rheumatoid arthritis by the managed use of each of the compositions (e.g., the CBD 5:1 salve and the 1:3 salve compositions) as needed for effective pain treatment.

Patient G was diagnosed with Neuromyelitis Optica Spectrum Disorder at the age of 15. Once leaving the hospital, Patient G was placed on several prescription pain medications to treat the nerve damage experienced following her latest attack of the disorder. These medications caused fatigue and decreased her overall quality of life. Patient G experienced muscle spasms and tightness in her joints. As an alternative to the prescription pain medications, Patient G topically applied a composition as described above, e.g., CBD 5:1 salve, to the skin on her back and shoulders where she had the most intense pain. About 2 weeks after topical application of the 5:1 product, Patient G experienced relief from the pain. Continued application of the salve composition resulted in sustained improvement in pain. Patient G reported that the effectiveness of the salve composition in treating and relieving her pain allowed her to eliminate taking completely one prescription pain medication and to reduce the dose of a second prescription pain medication from 1800 mg to 600 mg per day. Patient G, who is now 17 years old, regained her quality of life since she began to use, and as she continues to use, the topical composition, which is considered by Patient G as “a life changing medicine”.

1. A composition for treating or healing a skin or neurological condition or disorder, comprising an effective amount of at least one analgesic component and at least one cannabis component, and a pharmaceutically acceptable carrier, excipient, or diluent.

2. The composition of claim 1, wherein the at least one analgesic component comprises one or more of capsaicin, sesquiterpene lactones and eucalyptol.

3. The composition of claim 1, wherein the at least one cannabis component is a cannabinoid, a cannabinol, or a cannabidiol.

4. The composition of claim 3, wherein the cannabinoid is (−)-trans-cannabidiol (CBD), tetrahydrocannabinol (THC), or a combination thereof.

5. The composition of claim 4, wherein the ratio of CBD:THC is 5:1 or 1:3; and, optionally, wherein the composition is in the form of a salve or balm.

6. The composition of claim 1, further comprising Vitamin A, Vitamin B1, Vitamin B2, Vitamin B6, Vitamin C, Vitamin E, terpinen-4-ol and 1,8 cineole, carotenoids, triterpenic alcohols, or a combination thereof.

7. (canceled)

8. The composition of claim 1, further comprising one or more of isopropyl palmitate, soy lecithin, oleic acid, shea nut butter, beeswax, sunflower oil, lanolin, olive oil, antimicrobials, or antivirals, and, optionally, wherein the composition further comprises seabuckthorn oil, capsicum oil, arnica, tea tree, eucalyptus, calendula, vitapherol T-50, and Optiphen ND.

9. (canceled)

10. A composition for treating or healing a skin or neurological condition or disorder, comprising a pharmaceutically effective amount of at least one antioxidant component, an avenanthramide component and at least one cannabis component, and a pharmaceutically acceptable carrier, excipient, or diluent.

11. The composition of claim 10, wherein the at least one cannabis component is a cannabinoid, a cannabinol, or a cannabidiol.

12. The composition of claim 11, wherein the cannabinoid is (−)-trans-cannabidiol (CBD), tetrahydrocannabinol (THC), or a combination thereof.

13. The composition of claim 12, wherein the ratio of CBD:THC is 1:1.

14. The composition of claim 10, further comprising sesame oil, hydrolyzed oats, D-orientine S, collagen, aloe vera, antioxidants, polyphenols, Vitamin E, Vitamin B3, terpenes, flavonoids, antimicrobials, or a combination thereof, and, optionally, wherein the composition further comprises one or more of isopropyl palmitate, soy lecithin, oleic acid, shea nut butter, beeswax, jojoba oil, ceteryl alcohol, glyceryl stearate, and Optiphen ND, and is in the form of a liniment.

15.-16. (canceled)

17. A method of treating a subject suffering from or at risk of a skin condition, acute injury, inflammation, pain, skin allergy, muscle spasms, or skin aging, the method comprising topically administering to the subject an effective amount of the composition of claim 1; and, optionally, wherein the subject suffers from shingles, arthritis, fibromyalgia, migraine, neuropathy, back pain, sprains, muscle pain, cartilage pain, acne, insect bite, skin rash, skin wound, or a combination thereof.

18. (canceled)

19. A method of treating a subject suffering from or at risk of from a skin condition, damaged skin, inflammation, aging skin, wrinkling skin, sunburn, pain, or a combination thereof, comprising topically administering to the subject an effective amount of the composition of claim 10; and, optionally, wherein the subject is suffering from acne, acne vulgaris, psoriasis, eczema, hyperpigmentation, or a combination thereof.

20.-23. (canceled)

24. A composition for topical or transdermal application to the skin or body, comprising an effective amount of a cannabinoid selected from THC, THCa, THCV, CBN, CBC, CBD, CBG, or a combination thereof, and a pharmaceutically acceptable carrier, excipient, or diluent.

25. (canceled)

26. The composition of claim 24, wherein the cannabinoid is present in an amount of about 0.2 mg to about 49 mg.

27. The composition of claim 24, which is a moisturizer, a salve, a lotion, or a lubricant.

28. The composition of claim 24, wherein the cannabinoids comprise THC, CBD and CBG and are present in the composition in an amount of about 1 mg to about 18 mg.

29.-30. (canceled)

31. The composition of claim 24, wherein the cannabinoids comprise THC and CBG and are present in the composition in an amount of about 3 mg to about 117 mg.

32.-33. (canceled)

34. The composition of claim 24, further comprising a terpene or cannabis derived terpene selected from α-pinene, ß-myrcene, D-limonene, terpinolene, or a combination thereof.

35. (canceled)

36. The composition of claim 24, further comprising shea nut butter, beeswax, sunflower oil, lanolin, olive oil, seabuckthorn oil, capsicum oil, oleic acid, arnica, tea tree, eucalyptus, isopropyl palmitate, soy lecithin, optiphen ND, calendula, medical cannabis, vitapherol T-50, or a combination thereof and, optionally further comprising Shea butter, Sesame oil, Jojoba oil, Hydrolyzed oats, Ceteryl alcohol, D'Orientine S, Syn Coll, Oleic acid, Olivem, Aloe vera liquid, Optiphen ND, Antioxidant complex T5, Glyceryl stearate, Vitapherole T-50, Niacinamide, Isopropyl palmitate, Soy lecithin, Medical cannabis, Palmarosa, Grapefruit, or a combination thereof.

37.-38. (canceled)

39. A method of treating a subject suffering from or at risk of from a skin condition, skin injury, damaged skin, pain, inflammation, or a combination thereof, comprising topically administering to the subject an effective amount of the composition of claim 1.

40. A method of treating a subject suffering from or at risk of from a skin condition, skin injury, damaged skin, pain, inflammation, or a combination thereof, comprising topically administering to the subject an effective amount of the composition of claim 24.

41.-44. (canceled)

45. The method of claim 17, wherein the subject is suffering from or at risk of pain associated with a disease or disorder selected from arthritis, Rheumatoid arthritis, diabetic neuropathy, sciatica, Ankylosing Spondylitis, or Neuromyelitis Optica Spectrum Disorder.

46. (canceled)
